(19) United States
(12) Patent Application Publication
(10) Pub. No.: US 2017/0281847 A1
(43) Pub. Date: Oct. 5, 2017

(54) REGENERATIVE PERITONEAL DIALYSIS SYSTEM

(71) Applicant: Medtronic, Inc., Minneapolis, MN (US)

(72) Inventors: VenKatesh R. Manda, Stillwater, MN (US); Martin T. Gerber, Maple Grove, MN (US); Christopher M. Hobot, Rogers, MN (US); David B. Lura, Maple Grove, MN (US); Thomas E. Meyer, Stillwater, MN (US)

(21) Appl. No.: 15/478,576

(22) Filed: Apr. 4, 2017

Related U.S. Application Data
(60) Provisional application No. 62/318,183, filed on Apr. 4, 2016.

(51) Int. Cl. A61M 1/28; A61L 2/00; A61K 31/715; A61K 31/198; A61K 33/00; A61K 31/7004

(52) U.S. Cl. CPC A61M 1/282; A61M 1/287; A61K 33/00; A61K 31/7004; A61K 31/715; A61K 31/198; A61L 2/0047; A61L 2/0017; A61M 2209/10; A61M 2205/75; A61M 2205/36; A61M 2205/3337; A61M 2205/50; A61M 2205/502; A61L 2202/21

(57) ABSTRACT

Systems and methods of generating and regenerating peritoneal dialysate are provided. The systems and methods use a dialysate regeneration module, a sterilization module and concentrates to prepare peritoneal dialysate from used peritoneal dialysate or source water. An optional integrated cycler for direct infusion of the generated peritoneal dialysate is included. Optional dialysate storage containers are provided for storage of the peritoneal dialysate prior to use.

### CROSS-REFERENCE TO RELATED APPLICATIONS

This application claims the benefit of and priority to U.S. Provisional Patent Application No. 62/318,183 filed Apr. 4, 2016, the entire disclosure of which is incorporated by reference herein.

### FIELD OF THE INVENTION

The invention relates to devices, systems, and methods for generating a peritoneal dialysate having purity and sterility characteristics suitable for Peritoneal Dialysis (PD) and regenerating used fluid for subsequent use. The peritoneal dialysate can be generated or regenerated from water or used fluid of variable quality using a dialysate generation flow path containing a sterilization module. The sterilization module can be any one or more of an ultrafilter, Ultraviolet (UV) light source, microbial filter, dialyzer, and combinations thereof. The peritoneal dialysate generation system and related methods can automatically generate peritoneal dialysate fluid and deliver peritoneal dialysis therapy to a patient with an integrated cycler and regenerate a used fluid for subsequent use as fresh peritoneal dialysate.

### BACKGROUND

Peritoneal Dialysis (PD), including Automated Peritoneal Dialysis (APD) and Continuous Ambulatory Peritoneal Dialysis (CAPD), is a dialysis treatment that can be performed at home, either by the patient alone or with a care-giver. PD differs from Hemodialysis (HD) in that blood is not removed from the body and passed through a dialyzer, but rather a catheter is placed in the peritoneal cavity and dialysate introduced directly into the peritoneal cavity. Blood is cleaned inside the patient using the patient's own peritoneum as a type of dialysis membrane. However, because fluid is directly introduced into a human body, the fluid used for peritoneal dialysate is generally required to be free of biological and chemical contaminants. The peritoneal dialysate should also contain specified concentrations of solutes buffer, osmotic agent, and cations for biocompatibility and for performing membrane exchange.

Peritonitis is a serious and common problem in the PD population that results in abdominal pain, fever, and cloudy dialysate. Peritonitis remains a leading complication of PD with around 18% of infection-related mortality in PD patients resulting from peritonitis (Fried et al., “Peritonitis influences mortality in peritoneal dialysis patients,” J. Am. Soc. Nephrol. 1996; 7:2176-2182). Moreover, peritonitis is a contributing factor to death in 16% of deaths on PD, and remains a major cause for patients discontinuing PD and switching to HD. Peritonitis and other peritoneal dialysis complications can often be traced to non-sterile techniques and/or contaminated starting dialysate.

The US FDA regulates pre-packaged dialysate for use in PD as a Class II drug if the pre-packaged dialysate is used in either a semi-automatic PD system or an automatic PD system (e.g., cycler system). See 21 C.F.R. Sec. 876.5630. If the peritoneal dialysate is not pre-packaged, the US FDA requires the dialysate be prepared from a dialysate concentrate and “sterile purified water,” which is defined by the FDA in 21 C.F.R. Sec. 165.110(a)(2)(iv) and (vii). Some possible contaminants present in water used to prepare dialysis fluid can be (i) particles, (ii) chemicals, and (iii) microbial contaminants such as bacteria, fungi and yeasts, and microbial derivatives or fragments (e.g., endotoxins released during active growth and lysis of micro-organisms). In additional to meeting purity and sterility requirements, peritoneal dialysate must also contain specific and precise amounts of solutes, such as sodium chloride, sodium bicarbonate, and cation infusates.

Because traditional peritoneal dialysis systems require FDA-approved, pre-packaged dialysate, the dialysate can be expensive due to high manufacturing, shipping, and storage costs. Shortages can also occur. The problems are not mitigated by on-site dialysate preparation because the source water must still meet high fluid purity and sterility characteristics. Such standards may be difficult to meet, particularly for continuous, automatic peritoneal dialysis machines designed for home use. Further, traditional systems usually require storage of hundreds of liters of dialysate bags, including 300 L or more of peritoneal dialysate and over 300 kg of fluid per month. Storage and shipping of the peritoneal dialysate is expensive, labor intensive, and requires significant storage space.

Known systems and methods require significant space to store peritoneal dialysate prior to use. Continuous ambulatory peritoneal dialysis (CAPD) traditionally uses 1-4 exchanges of peritoneal dialysate a day, with an overnight dwell. Because each exchange requires approximately 2-4 L of peritoneal dialysate, use of prepackaged dialysate requires storing about 8-16 L of dialysate per day, or 56-112 L of dialysate per week. Automated peritoneal dialysis uses a cycler to cycle peritoneal dialysis into and out of the peritoneal cavity of the patient, generally at night. APD generally uses 3-5 exchanges daily, requiring up to 20 L of dialysate per day and up to 140 L of dialysate per week. Tidal Peritoneal Dialysis (TPD) is similar to APD with the exception that a between 250 mL to 1000 mL of the peritoneal dialysate is left in the peritoneal cavity of the patient between infusions. As such, the known treatments require significant amounts of clean water, which can deter using known systems, particularly in areas where clean water is scarce.

Hence, there is a need for systems and methods that can regenerate and reuse peritoneal dialysate after a first treatment, lowering the requirements for fresh water and storage space. There is also a need for a system that can regenerate used peritoneal dialysate and reuse the peritoneal dialysate with an integrated cycler, reducing the number of components necessary for peritoneal dialysis.

### SUMMARY OF THE INVENTION

The first aspect of the invention relates to a system. In any embodiment, the system can include a water source; a peritoneal dialysate generation flow path; wherein the peritoneal dialysate generation flow path is fluidly connectable to the water source; one or more peritoneal dialysate regeneration modules fluidly connectable to the peritoneal dialysate generation flow path; a concentrate source fluidly connectable to the peritoneal dialysate generation flow path; the concentrate source containing one or more solutes; and a sterilization module fluidly connectable to the peritoneal dialysate generation flow path.

In any embodiment of the first aspect of the invention, the peritoneal dialysate generation flow path can include connectors for connection to a cycler.

In any embodiment of the first aspect of the invention, the system can include an integrated cycler; the integrated cycler having a pump, an infusion line, and a drain line; wherein the infusion line is fluidly connected to the peritoneal dialysate generation flow path downstream of the sterilization module; and wherein the drain line is fluidly connected to the peritoneal dialysate generation flow path upstream of the peritoneal dialysate regeneration module.

In any embodiment of the first aspect of the invention, the system can have one or more dialysate containers fluidly connectable to the peritoneal dialysate generation flow path downstream of the sterilization module.

In any embodiment of the first aspect of the invention, the concentrate source can include one or more of an osmotic agent and an ion concentrate.

In any embodiment of the first aspect of the invention, the concentrate source can include at least an osmotic agent source and an ion concentrate source.

In any embodiment of the first aspect of the invention, the concentrate source can include multiple osmotic agent sources.

In any embodiment of the first aspect of the invention, the osmotic agent sources can contain osmotic agents selected from the group of dextrose, icodextrin, amino acids, and glucose.

In any embodiment of the first aspect of the invention, the ion concentrate source can include one or more from the group of sodium chloride, sodium lactate, magnesium chloride, calcium chloride, potassium chloride, and sodium bicarbonate.

In any embodiment of the first or second aspect of the invention, the concentrate source can include multiple ion concentrate sources.

In any embodiment of the first aspect of the invention, the system can have a concentrate pump positioned between the concentrate source and the peritoneal dialysate generation flow path for controlled addition of fluid from the concentrate source to the peritoneal dialysate generation flow path.

In any embodiment of the first aspect of the invention, the system can have a control system for controlling one or more pumps and valves to control movement of fluid through the system.

In any embodiment of the first aspect of the invention, the control system can include a timer wherein the timer causes the control system to generate peritoneal dialysate at a predetermined time.

In any embodiment of the first aspect of the invention, the control system can include a user interface, wherein the user interface causes the control system to generate peritoneal dialysate at a selected time.

In any embodiment of the first aspect of the invention, the sterilization module can include one or more ultrafilters; a UV light source; a heater, a flash pasteurization module, a microbial filter; or combinations thereof.

In any embodiment of the first aspect of the invention, the sterilization module can include the UV light source positioned downstream of the ultrafilter.

In any embodiment of the first aspect of the invention, the peritoneal dialysate regeneration module can include one or more selected from the group of a sorbent cartridge, activated carbon, an ion exchange resin, a reverse osmosis module, a carbon filter, and a nanofilter.

In any embodiment of the first aspect of the invention, the integrated cycler can have a heater.

In any embodiment of the first aspect of the invention, the integrated cycler can have at least one sensor selected from the group of a flow meter, a pressure sensor, a conductivity sensor, and a temperature sensor.

In any embodiment of the first aspect of the invention, the system can have a second ultrafilter in the peritoneal dialysate generation flow path.

In any embodiment of the first aspect of the invention, the integrated cycler can include a filter in the infusion line.

The features disclosed as being part of the first aspect of the invention can be in the first aspect of the invention, either alone or in combination, or follow a preferred arrangement of one or more of the described elements.

The second aspect of the invention is drawn to a method. In any embodiment of the second aspect of the invention, the method can include the steps of pumping fluid through a peritoneal dialysate generation flow path having a peritoneal dialysate regeneration module; adding one or more concentrate solutions to the fluid in the peritoneal dialysate generation flow path; and pumping the fluid through a sterilization module.

In any embodiment of the second aspect of the invention, the fluid can be fluid returned to the peritoneal dialysate generation flow path from a peritoneal cavity of a patient.

In any embodiment of the second aspect of the invention, the method can include heating the fluid; pumping the fluid into a peritoneal cavity of a patient with an integrated cycler; and pumping the fluid from the peritoneal cavity of the patient into the peritoneal dialysate generation flow path.

In any embodiment of the second aspect of the invention, the method can include the step of pumping the fluid into one or more dialysate containers and pumping the fluid from the one or more dialysate containers into the peritoneal cavity of the patient.

In any embodiment of the second aspect of the invention, the step of adding one or more concentrate solutions to the fluid can include adding at least an osmotic agent and an ion concentrate to the fluid.

In any embodiment of the second aspect of the invention, the osmotic agent and ion concentrate can be added to the fluid from a single concentrate source.

In any embodiment of the second aspect of the invention, the osmotic agent and ion concentrate can be added from separate concentrate sources.

In any embodiment of the second aspect of the invention, the osmotic agent can be one or more selected from the group of glucose, dextrin, and icodextrin.

In any embodiment of the second aspect of the invention, the osmotic agent can include multiple osmotic agents.

In any embodiment of the second aspect of the invention, the multiple osmotic agents can be added from a single osmotic agent source.

In any embodiment of the second aspect of the invention, each of the multiple osmotic agents can be added from separate osmotic agent sources.

In any embodiment of the second aspect of the invention, the ion concentrate can be added from an ion concentrate source and can include one or more of sodium chloride, sodium lactate, magnesium chloride, calcium chloride, potassium chloride, and sodium bicarbonate.

In any embodiment of the second aspect of the invention, each of the ion concentrates can be added to the fluid from a single ion concentrate source.

In any embodiment of the second aspect of the invention, the ion concentrate source can include multiple ion concentrate sources; and each of the multiple ion concentrate sources can include different solutes.

In any embodiment of the second aspect of the invention, the step of adding one or more concentrate solutions to the fluid can include controlling an addition of concentrate from each of the ion concentrate sources to generate a peritoneal dialysate with a prescribed solute concentration.

In any embodiment of the second aspect of the invention, the method can be carried out by a peritoneal dialysate generation system having a control system.

In any embodiment of the second aspect of the invention, the peritoneal dialysate regeneration module can include one or more of a sorbent cartridge, activated carbon, a reverse osmosis module, a carbon filter and a nanofilter.

In any embodiment of the second aspect of the invention, the sterilization module can include one or more ultrafilters; a UV light source; a microbial filter; or combinations thereof.

In any embodiment of the second aspect of the invention, the sterilization module can include at least two ultrafilters.

The features disclosed as being part of the second aspect of the invention can be in the second aspect of the invention, either alone or in combination, or follow a preferred arrangement of one or more of the described elements.

### BRIEF DESCRIPTION OF THE DRAWINGS

FIG. 1 shows a flow schematic of a regenerative peritoneal dialysate system.

FIG. 2 shows a detailed flow diagram of a regenerative peritoneal dialysate system.

FIG. 3 shows a detailed flow diagram for a regenerative peritoneal dialysate system with an integrated cycler.

FIG. 4 shows a system for adding concentrates to a peritoneal dialysate generation flow path.

FIG. 5 shows an overview of a system for generating, regenerating, and using peritoneal dialysate with a single concentrate source.

FIG. 6 shows an overview of a system for generating, regenerating, and using peritoneal dialysate with multiple concentrate sources.

FIG. 7A shows a perspective view of a peritoneal dialysate generation cabinet.

FIG. 7B shows a front view of a peritoneal dialysate generation cabinet.

FIG. 8 shows a perspective view of a peritoneal dialysate generation cabinet with door panels closed.

FIGS. 9A-D show a peritoneal dialysate generation cabinet with a water reservoir and waste reservoir.

FIG. 10 shows a peritoneal dialysate generation cabinet connected to a faucet and drain.

FIG. 11 shows a dialysis caddy for use in a peritoneal dialysate generation flow path.

### DETAILED DESCRIPTION OF THE INVENTION

Unless defined otherwise, all technical and scientific terms used have the same meaning as commonly understood by one of ordinary skill in the art.

The articles “a” and “an” are used to refer to one or to over one (i.e., to at least one) of the grammatical object of the article. For example, “an element” means one element or over one element.

“Activated carbon” refers to a form of carbon processed to have small pores, increasing the surface area available for adsorption.

The term “amino acid,” as used herein, refers to any nitrogen containing organic acid or peptide that can be used as an osmotic agent to generate peritoneal dialysate.

The term “calcium chloride source” refers to a source of calcium chloride in solid and/or solution form. The calcium chloride source can contain at least one fluid pathway and include components such as conduits, valves, filters or fluid connection ports, any of which are fluidly connectable to each other or to a fluid flow path. The calcium chloride source can either be formed as a stand-alone enclosure or a compartment integrally formed with an apparatus for dialysis for containing a calcium chloride source.

A “carbon filter” is a bed of activated carbon.

The term “comprising” includes, but is not limited to, whatever follows the word “comprising.” Use of the term indicates the listed elements are required or mandatory but that other elements are optional and may be present.

A “concentrate pump” is a pump configured to move fluid between a concentrate source and a flow path.

A “concentrate solution” is a solution of one or more solutes in water. The concentrate solution can have a solute concentration greater than that to be used in dialysis.

A “concentrate source” is a source of one or more solutes. The concentrate source can have one or more solutes that has a solute concentration greater than the solute concentration to be used for dialysis.

A “conductivity sensor” is device for measuring the electrical conductance of a solution and/or the ion, such as a sodium ion, concentration of a solution.

A “connector” and “for connection” describe the concept of forming a fluid connection between two components wherein fluid, gas, or mixture of both gas and fluid can flow from one component, through a connector or a component for connection, to another component. The connector provides for a fluid connection in its broadest sense and can include any type of tubing, fluid or gas passageway, or conduit between any one or more components of the invention. The connection can optionally be disconnected and then reconnected.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts or features that do not affect the basic operation of the apparatus, structure or method described.

The terms “control,” “controlling,” or “controls” refers to the ability of one component to direct the actions of a second component.

A “control system” can be a combination of components that act together to maintain a system to a desired set of performance specifications. The control system can use processors, memory and computer components configured to interoperate to maintain the desired performance specifications. The control system can also include fluid or gas control components, and solute control components as known within the art to maintain the performance specifications.

The terms “controlled addition,” to “control addition,” or “controlling addition” refer to the ability to add one or more substances or fluids to a flow path or container in an accurately controllable amount.

The phrase “controlling the movement of fluid” refers to directing fluid through a flow path, container, receptacle, or reservoir of any type.

A “cycler” is a component for movement of fluid into and out of the peritoneal cavity of a patient.

The term “dextrose source” refers to a source of dextrose in solid and/or solution form. The dextrose source can interface with at least one other module found in systems for dialysis. The dextrose source can contain at least one fluid pathway and include components such as conduits, valves, filters or fluid connection ports, any of which are fluidly connectable to each other or to a fluid flow path. The dextrose source can either be formed as a stand-alone enclosure or a compartment integrally formed with an apparatus for dialysis for containing a dextrose source.

The term “dialysate” describes a fluid into or out of which solutes from a fluid to be dialyzed diffuse through a membrane. Dialysate can differ depending on the type of dialysis to be carried out. For example, dialysate for peritoneal dialysis may include different solutes or different concentrations of solutes than dialysate for hemodialysis.

A “dialysate container” is any container capable of storing or containing dialysate for dialysis. The container any be of any suitable, size, geometry, or configuration.

The term “dialysis caddy” refers to a container detachably removable from a dialysis system, the caddy configured to hold one or more other containers. In any embodiment, the caddy can include one or more connectors for fluid connection from the containers to the dialysis system.

The term “downstream” refers to a position of a first component in a flow path relative to a second component wherein fluid will pass by the second component prior to the first component during normal operation. The first component can be said to be “downstream” of the second component, while the second component is “upstream” of the first component.

A “drain line” is a fluid line for carrying fluid to a drain such as a waste receptacle or drain. The drain line can be connected to a peritoneal cavity of a patient for draining fluid.

The term “filter” refers to a porous component through which fluid can pass, but that traps one or more materials within the fluid.

A “fitting feature” is any protrusion, indentation, groove, ridge, having any shape, size, or geometry that ensures that only a corresponding fitting feature complementary to the fitting feature can form a connection or fit to the corresponding fitting feature. The fitting feature can also include non-mechanical means for ensuring complementary connection such as magnets placed at particular locations, or visual or aural indicators such as color, lettering, or sound. The fitting feature can be affixed, integral, or labeled on a component or surface to ensure that a corresponding feature on a desired component or surface can mate or connect to the component or surface having the fitting feature.

A “flash pasteurization module” is a component or set of components capable of heating a fluid to a high temperature and recirculating the fluid for sterilization.

A “flow meter” is a device capable of measuring an amount or rate of fluid moving past or through a particular location.

The term “fluid” can be any substance that has no fixed shape that yields easily to external pressure such as a gas or a liquid. Specifically, the fluid can be water containing any solutes at any concentration. The fluid can also be dialysate of any type including fresh, partially used, or spent.

The terms “fluid connection,” “fluidly connectable,” or “fluidly connected” refer to the ability to pass fluid or gas from one point to another point. The two points can be within or between any one or more of compartments, modules, systems, and components, all of any type.

“Fluid returned to the peritoneal dialysate generation flow path from a peritoneal cavity of a patient” refers to fluid used in peritoneal cavity and then returned to the peritoneal dialysate generation flow path.

The terms “to generate peritoneal dialysate” or “peritoneal dialysate generation” refers to creating a peritoneal dialysate solution from constituent parts.

The term “glucose source” refers to a source of glucose in solid and/or solution form. The glucose source can interface with at least one other module found in systems for dialysis. The glucose source can contain at least one fluid pathway and include components such as conduits, valves, filters or fluid connection ports, any of which are fluidly connectable to each other or to a fluid flow path. The glucose source can either be formed as a stand-alone enclosure or a compartment integrally formed with an apparatus for dialysis for containing a glucose source.

A “heater” is a component capable of raising the temperature of a substance, container, or fluid.

The terms “heating” or to “heat” refer to raising the temperature of a substance, fluid, or container.

An “integrated cycler” is a component for movement of fluid into and out of the peritoneal cavity of a patient, wherein the integrated cycler forms a part of an overall system. For example, the integrated cycler can be contained in a housing with other components used for peritoneal dialysis and be in fluid and electrical connection with desired components.

An “infusion line” is a fluid line for carrying peritoneal dialysate into a body cavity or part of a patient such as a peritoneal cavity.

An “ion concentrate” refers to one or more ionic compounds. The ion concentrate can have one or more ionic compounds in the ion concentrate. Further, the ion concentrate can have an ion concentration greater than an ion concentration to be used in dialysis.

An “ion concentrate source” refers to a source of one or more ionic compounds. The ion concentrate source can be in water or solid form. The ion concentrate source can further have one or more ionic compounds that is at a higher ion concentration greater than generally used in dialysis.

The term “ion exchange resin” refers to a material capable of removing ions from a fluid and releasing different ions into the fluid.

The term “level of sterility” refers to an estimated probability of viable organisms surviving a sterilization process.

The term “magnesium chloride source” refers to a source of magnesium chloride in solid and/or solution form. The magnesium chloride source can interface with at least one other module found in systems for dialysis. The magnesium chloride source can contain at least one fluid pathway and include components such as conduits, valves, filters or fluid connection ports, any of which are fluidly connectable to each other or to a fluid flow path. The magnesium chloride source can either be formed as a stand-alone enclosure or a compartment integrally formed with an apparatus for dialysis for containing a magnesium chloride source.

The term “microbial filter” refers to a device inhibiting passage of microbes or fragments of microbes such as endotoxins in a fluid or solution while allowing the passage of the fluid or solution.

A “nanofilter” is a filter membrane having nanometer sized pores.

An “osmotic agent” is a substance dissolved in water capable of driving a net movement of water by osmosis across a semi-permeable membrane due to concentration differences of the osmotic agent on each side of the semi-permeable membrane.

An “osmotic agent source” refers to a source of osmotic agents in solid and/or solution form. The osmotic agent source can interface with at least one other module found in systems for dialysis. The osmotic agent source can contain at least one fluid pathway and include components such as conduits, valves, filters or fluid connection ports, any of which are fluidly connectable to each other or to a fluid flow path. The osmotic agent source can either be formed as a stand-alone enclosure or a compartment integrally formed with an apparatus for dialysis for containing an osmotic agent source.

The term “peritoneal cavity” refers to the space between the parietal peritoneum and visceral peritoneum of a patient.

“Peritoneal dialysate” is a dialysis solution that can to be used in peritoneal dialysis having specified parameters for purity and sterility. Peritoneal dialysate is not the same as dialysate used in hemodialysis although peritoneal dialysate may be used in hemodialysis.

A “peritoneal dialysate generation flow path” is a path that can be used in generating dialysate suitable for peritoneal dialysis.

A “peritoneal dialysate generation system” refers to a collection of components used to generate peritoneal dialysate.

The term “peritoneal dialysate regeneration module” refers to a component or components capable of removing waste products from a fluid.

“Peritoneal dialysis” is a therapy wherein a dialysate is infused into the peritoneal cavity, which serves as a natural dialyzer. In general, waste components diffuse from a patient's bloodstream across a peritoneal membrane into the dialysis solution via a concentration gradient. In general, excess fluid in the form of plasma water flows from a patient's bloodstream across a peritoneal membrane into the dialysis solution via an osmotic gradient. Once the infused peritoneal dialysis solution has captured sufficient amounts of the waste components the fluid is removed. This cycle can be repeated for several cycles each day or as needed.

A “predetermined time” is a set time for an event to occur, such as a set time of day, or a set length of time from a previous event.

The term “prescribed solute concentration” refers to the concentration of one or more solutes in peritoneal dialysate intended for use by a patient.

The term “pressure sensor” refers to a device for measuring the pressure of a gas or liquid in a vessel, container, or fluid line.

The term “pump” refers to any device that causes the movement of fluids or gases by applying suction or pressure.

The terms “pumping fluid” or to “pump fluid” refer to moving a fluid through a flow path with a pump.

“Purified water” can be defined as water produced by distillation, deionization, reverse osmosis, or other suitable processes and that meets the definition of “purified water” in the United States Pharmacopeia, 23d Revision, Jan. 1, 1995, and the FDA at 21 CFR Section §165.110(a)(2)(iv). Other criteria for purified water can be determined by those of skill in the art, particularly as relating to purified water suitable for peritoneal dialysis.

The terms “regenerative peritoneal dialysis” or to “regenerate peritoneal dialysate” refer to removing waste products from used peritoneal dialysate to generate a fluid reusable in peritoneal dialysis.

A “reverse osmosis module” is a set of components that act to drive fluid through one or more semipermeable membranes, wherein pressure is used to move the fluid from a side of the semipermeable membrane with a higher concentration of one or more solutes to a side of the semipermeable membrane with a lower concentration of the one or more solutes.

A “selected time” is a set time chosen by a user or algorithm.

A “sensor” is a component capable of determining or sensing the states of one or more variables in a system.

The term “sodium chloride source” refers to a source of sodium chloride in solid and/or solution form. The sodium chloride source can interface with at least one other module found in systems for dialysis. The sodium chloride source can contain at least one fluid pathway and include components such as conduits, valves, filters or fluid connection ports, any of which are fluidly connectable to each other or to a fluid flow path. The sodium chloride source can either be formed as a stand-alone enclosure or a compartment integrally formed with an apparatus for dialysis for containing a sodium chloride source.

The term “sodium lactate source” refers to a source of sodium lactate in solid and/or solution form. The sodium lactate source can interface with at least one other module found in systems for dialysis. The sodium lactate source can contain at least one fluid pathway and include components such as conduits, valves, filters or fluid connection ports, any of which are fluidly connectable to each other or to a fluid flow path. The sodium lactate source can either be formed as a stand-alone enclosure or a compartment integrally formed with an apparatus for dialysis for containing a sodium lactate source.

A “solute” is a substance dissolved in a solvent, such as water.

The term “sorbent cartridge” refers to a cartridge containing one or more sorbent materials for removing specific solutes from solution. The term “sorbent cartridge” does not require the contents in the cartridge be sorbent based, and the contents of the sorbent cartridge can be any contents that can remove solutes from a dialysate. The sorbent cartridge may include any suitable amount of one or more sorbent materials. In certain instances, the term “sorbent cartridge” refers to a cartridge which includes one or more sorbent materials besides one or more other materials capable of removing solutes from dialysate. “Sorbent cartridge” can include configurations where at least some materials in the cartridge do not act by mechanisms of adsorption or absorption.

A “sterilization module” is a component or set of components to sterilize a fluid by removing or destroying chemical or biological contaminants.

A “temperature sensor” is a sensor capable of determining the temperature of a fluid.

A “timer” is a device capable of determining the time of day, or the time elapsed between multiple events.

An “ultrafilter” is a semi permeable membrane through which a fluid can pass and that can remove one or more solutes or particles from the fluid.

The term “upstream” refers to a position of a first component in a flow path relative to a second component wherein fluid will pass by the first component prior to the second component during normal operation. The first component can be said to be “upstream” of the second component, while the second component is “downstream” of the first component.

A “user interface” is a component that allows a user to communicate information or instructions to a processor or a memory device and to receive information or instructions from the processor or memory device.

A “UV light source” is a component that uses ultraviolet light to kill biological contaminants in a fluid.

A “valve” is a device capable of directing the flow of fluid or gas by opening, closing or obstructing one or more pathways to allow the fluid or gas to travel in a path. One or more valves configured to accomplish a desired flow can be configured into a “valve assembly.”

The term “water purification module” refers to a component or components capable of removing biological or chemical contaminants from water.

The term “water source” refers to a source from which potable or not potable water can be obtained.

### Regenerative Peritoneal Dialysis System

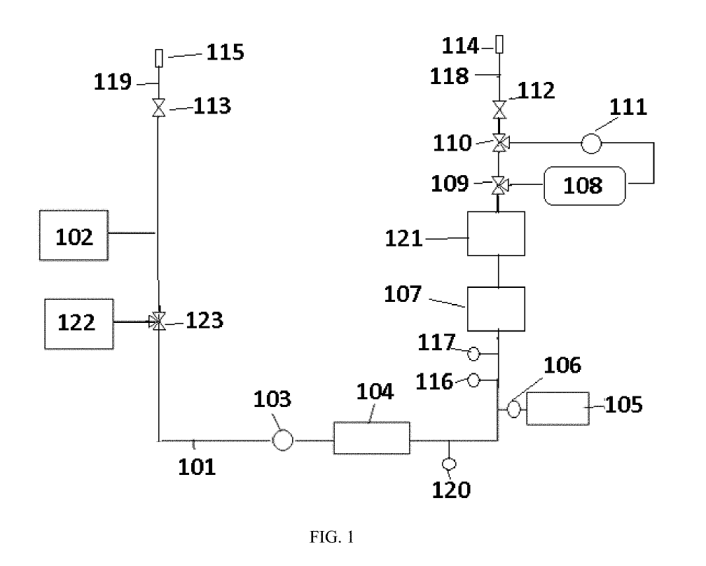

The invention is drawn to systems and methods for regenerating and reusing peritoneal dialysate. FIG. 1 illustrates a block diagram of a flow path used in regenerating peritoneal dialysate. The flow path includes a peritoneal dialysate generation flow path 101. Water source 102 can provide water for the initial preparation of peritoneal dialysate, as well as additional fluid as needed. System pump 103 provides a driving force for moving fluid through the peritoneal dialysate generation flow path 101. The fluid in the peritoneal dialysate generation flow path 101 is pumped through a peritoneal dialysate regeneration module 104 to remove waste products and impurities. The water source 102 can be a source of potable water including a purified water source. Purified water can refer to water meeting the definition of “purified water” in the United States Pharmacopeia, 23d Revision, Jan. 1, 1995. Alternatively, purified water can refer to any source of water treated to remove at least some biological or chemical contaminants, whether or not the water meets the definition of purified water in United States Pharmacopeia, 23d Revision, Jan. 1, 1995. In any embodiment of the first or second aspects of the invention, the water source 102 can be a non-purified water source, such as tap water, wherein the water from the water source 102 can be purified by the system as described. A non-purified water source can provide water without additional purification, such as tap water from a municipal water source, water that has undergone some level of purification, but does not meet the definition of “purified water” provided, such as bottled water or filtered water. In any embodiment, the water source can contain water meeting the WHO drinkable water standards provided in Guidelines for Drinking Water Quality, World Health Organization, Geneva, Switzerland, 4th edition, 2011. The water source 102 can be any size usable with the system, including between 3 and 20 L. A water source 102 of 6 L or less can generally generate the necessary peritoneal dialysate for multiple cycles when using a regenerative system. The peritoneal dialysate generation flow path 101 can also have a direct connection to a purified or non-purified water source. The water source 102 can be any source of water, whether from a tap, faucet, or a separate container or reservoir.

In any embodiment of the first or second aspects of the invention, the peritoneal dialysate regeneration module 104 can be a sorbent cartridge. The sorbent cartridge includes urease, a cation exchange material, an anion exchange material, and activated carbon. The urease can optionally be immobilized on alumina. The activated carbon operates to adsorb non-ionic molecules, organic molecules, and chlorine from the water, along with some endotoxins or bacterial contaminants. The activated carbon can be present in the sorbent cartridge in the form of a carbon filter or pad, or as a material layer in the sorbent cartridge. A carbon filter or pad is a bed of activated carbon. The carbon filter can be in a self contained packaging, or present as a layer of activated carbon within the sorbent cartridge. The urease catalyzes the conversion of urea in the dialysate into ammonium ions. The cation exchange material, such as zirconium phosphate, can remove cationic species from the fluid, such as potassium, calcium, magnesium, the ammonium ions generated by the reaction of urea, or other cations, replacing the cationic species with hydrogen or sodium. The sorbent cartridge can include any cation exchange material capable of removing cations from the fluid. The anion exchange material, such as zirconium oxide, can remove anionic species from the fluid, such as phosphate or fluoride molecules, replacing the anionic species with acetate or hydroxide ions. The sorbent cartridge can have any anion exchange material known in the art capable of removing anionic species from the fluid. In any embodiment, the sorbent cartridge can include aluminum oxide for removal of fluoride and heavy metals. In any embodiment, the sorbent cartridge can have a layer of aluminum oxide, followed by a layer of activate carbon, a layer of urease, and then an ion exchange resin. The sorbent cartridge can purify up to 3 L of water or used peritoneal dialysate per exchange, with flow rates of up to 300 ml/min. A larger sorbent cartridge can be used when generating peritoneal dialysate for multiple infusions, including a sorbent cartridge that can purify between 3 and 20 L, between 3 and 5 L, between 3 and 10 L, between 5 and 12 L, between 10 and 15 L, or between 10 and 20 L of water, or more.

In any embodiment, the sorbent cartridge can be a single use component or a rechargeable component. Recharging can refer to the process of treating a sorbent material to restore the functional capacity of the sorbent material so as to put the sorbent material back into a condition for use or reuse in a new dialysis session. In some instances, recharging also includes treating a sorbent material so as to clean the sorbent material so that the sorbent material can be stored and used in a subsequent dialysis session. In some instances, the total mass, weight and/or amount of “rechargeable” sorbent materials remain the same. In some instances, the total mass, weight and/or amount of “rechargeable” sorbent materials change. Without being limited to any one theory of invention, the recharging process may involve exchanging ions bound to the sorbent material with different ions, which in some instances may increase or decrease the total mass of the system. However, the total amount of the sorbent material will in some instances be unchanged by the recharging process. Upon a sorbent material undergoing “recharging,” the sorbent material can then be said to be “recharged.”

The sorbent cartridge can additionally include a microbial filter and/or a particulate filter. A microbial filter can further reduce the amount of endotoxins or bacterial contaminants present in the fluid. A particulate filter can remove particulate matter from the fluid. The sorbent materials described can be present in the sorbent cartridge in any order, or intermixed, so long as the zirconium phosphate is present downstream of the urease. The sorbent cartridge can be sized depending on the needs of the user, with a larger sized sorbent cartridge allowing for more exchanges before the sorbent cartridge must be replaced.

Alternatively, the peritoneal dialysate regeneration module 104 can be any component capable of removing contaminants from the fluid in the peritoneal dialysate generation flow path 101, including any one or more of a sorbent cartridge, reverse osmosis module, nanofilter, an ion exchange resin, a combination of cation and anion exchange materials, activated carbon, silica, or silica based columns.

After passing through the peritoneal dialysate regeneration module 104, the fluid is pumped to a concentrate source 105, where necessary components for carrying out peritoneal dialysis can be added from the concentrate source 105. The concentrates in the concentrate source 105 are utilized to create a peritoneal dialysis fluid that matches a dialysis prescription. Concentrate pump 106 can control the movement of concentrates from the concentrate source 105 to the peritoneal dialysate generation flow path 101 in a controlled addition. The concentrates added from the concentrate source 105 to the peritoneal dialysate generation flow path 101 can include any component prescribed for use in peritoneal dialysate. Table 1 provides non-limiting exemplary ranges of commonly used components of peritoneal dialysate.

TABLE 1

| Component | Concentration | |
|---|---|---|
| Sodium chloride | 132-134 | mmol/L |
| Calcium chloride dehydrate | 1.25-1.75 | mmol/L |
| Magnesium chloride hexahydrate | 0.25-0.75 | mmol/L |
| Sodium Lactate | 35-40 | mmol/L |
| Dextrose (D-glucose) monohydrate | 0.55-4.25 | g/dL |

| | |
|---|---|
| pH | 5-6 |
| Osmolality | 346-485 (hypertonic) |

To reduce the glucose degradation products (GDP) formed, some peritoneal dialysate systems use a low GDP formulation. Exemplary peritoneal dialysate concentrations for low GDP formulations are provided in Table 2. Generally, the low GDP peritoneal dialysate is provided in two separate bags, with one bag containing calcium chloride, magnesium chloride and glucose maintained at low pH, and the second bag containing sodium chloride and the buffer components, including sodium lactate and sodium bicarbonate. The two bags are mixed prior to use to generate a peritoneal dialysate with a neutral pH. Alternatively, a two chamber bag is used, the chambers separated by a divider which is broken to mix the fluids prior to use.

TABLE 2
Low GDP peritoneal dialysate formulations

| Component | Concentration | |
|---|---|---|
| Sodium | 132-134 | mEq/L |
| Calcium | 2.5-3.5 | mEq/L |
| Magnesium | 0.5-1.0 | mEq/L |
| Lactate | 0-40 | mEq/L |
| Bicarbonate | 0-34 | mEq/L |

| | |
|---|---|
| pH | 6.3-7.4 |
| % glucose (g/dL) | 1.5-4.25 |

One of skill in the art will understand that other components can be used in place of the components listed in Tables 1-2. For example, dextrose as listed in Table 1 is commonly used as an osmotic agent. In any embodiment of the first or second aspects of the invention, other osmotic agents can be used in addition to, or in place of, the dextrose, including glucose, icodextrin or amino acid solutions, including dialysate with multiple osmotic agents. Although the sources of sodium, calcium, and magnesium listed in Table 1 are chloride salts, other sodium, magnesium, and calcium salts can be used, such as lactate or acetate salts. Peritoneal dialysate may also contain buffers for maintaining pH of the peritoneal dialysate. Exemplary, non-limiting examples of suitable buffers include bicarbonate buffer, acetate buffer or lactate buffer. Although not generally used in peritoneal dialysis, potassium chloride can be used for hypokalemic patients who don't receive sufficient potassium through diet. The concentrate source 105 can contain one or more osmotic agents, as well as one or more ion concentrates, such as concentrated sodium chloride, sodium lactate, magnesium chloride, calcium chloride, and/or sodium bicarbonate. In any embodiment, the system can have a single concentrate that has all components mixed for a daytime or overnight treatment for use in a home by a single patient. The concentrate source 105 can include separate sources for any one or more of the solutes that are to be used in the peritoneal dialysate. Alternatively, the concentrate source 105 can include a separate osmotic agent source, and an ion concentrate source each with a separate concentrate pump to add each component needed to create the peritoneal dialysate. Concentrate pump 106 pumps concentrated solutions from the concentrate source or sources 105 to the peritoneal dialysate generation flow path 101 in a controlled addition. Where more than one concentrate source is used, separate concentrate pumps can move each of the concentrates into the peritoneal dialysate generation flow path 101, or a single concentrate pump can be used, with valves configured allow individual control over the movement of each of the concentrate solutions to the peritoneal dialysate generation flow path 101.

One of skill in the art will understand that the peritoneal dialysate regeneration module 104 may not fully remove glucose, dextrose, icodextrin, or other osmotic agents. To control the amount of osmotic agent in the generated dialysate, the relative amounts of ionic solutes and osmotic agents required to be added may vary. To control the relative amounts of ions and osmotic agents in the dialysate where the peritoneal dialysate regeneration module 104 does not fully remove the osmotic agents, the concentrate source 105 can include a separate osmotic agent source and ion concentrate source. Refractive index sensor 120 determines the concentration of glucose or other osmotic agents in the dialysate downstream of the peritoneal dialysate regeneration module 104 and can control the addition of osmotic agents from the concentrate source 105.

After addition of solutes from the concentrate source 105, the fluid in the peritoneal dialysate generation flow path 101 can contain all the necessary solutes for peritoneal dialysis. Conductivity sensor 116 and refractive index sensor 117 are used to confirm the concentration of electrolytes and osmotic agents are within predetermined ranges.

The peritoneal dialysate should reach a level of sterility for peritoneal dialysis. The level of sterility can be any level that meets an applicable regulatory requirement, such as a sterility assurance level of 10⁻⁶ required by the FDA, meaning that the chance a viable organism is present after sterilization is 1 in 1,000,000. The system can pump the fluid to a sterilization module for sterilization of the peritoneal dialysate. The sterilization module can include one or more of a first ultrafilter 107, a second ultrafilter 121, and a UV light source (not shown in FIG. 1). The sterilization module can be any component or set of components capable of sterilizing the peritoneal dialysate. In any embodiment, the sterilization module can be a single or multiple ultrafilters. Multiple ultrafilters provide further sterilization of the fluid and redundancy of the system to protect against sterilization failure. A secondary component, such as a UV light source or microbial filter (not shown in FIG. 1), can be used in the sterilization module to provide additional sterilization of the peritoneal dialysate. The UV light source can be positioned at any location in the peritoneal dialysate generation flow path 101, including upstream of the first ultrafilter 107, between ultrafilters 107 and 121 in a system using two ultrafilters, or downstream of the second ultrafilter 121. The ultrafilters 107 and 121 used in the sterilization module can be replaced as necessary. In any embodiment, the ultrafilters 107 and 121 can have a 3-6 month lifetime before replacement. The ultrafilters 107 and 121 can be any ultrafilter known in the art capable of sterilizing the peritoneal dialysate. A non-limiting example of an ultrafilter that can be used in the systems described is the hollow fiber ForClean ultrafilter, available from Bellco, Mirandola (MO), Italy. In certain embodiments, the sterilization module can use heat sterilization. The sterilization module can include a heater (not shown) to heat the prepared dialysate. Alternatively or additionally, the sterilization module can include a flash pasteurization module (not shown) to sterilize the dialysate through flash pasteurization. The sterilization module can include both heat-based sterilization components and filtration based sterilization components, with the user adjusting the mode of sterilization based on the mode of use. For example, a heat based sterilization can be used when the peritoneal dialysate is generated for later use, while a filtration based sterilization can be used when the peritoneal dialysate is generated for immediate use.

After passing through the sterilization module, the peritoneal dialysate can be used in peritoneal dialysis. A dialysate storage container 108 can store the peritoneal dialysate until used. The peritoneal dialysate can pass through valve 109 and into the dialysate storage container 108. If ready for use, the peritoneal dialysate can be pumped from dialysate storage container 108, through valve 110, and back into the peritoneal dialysate generation flow path 101. Pump 111 provides a driving force for the movement of the peritoneal dialysate from the dialysate storage container 108 to the peritoneal dialysate generation flow path 101. Additional or alternative storage containers can be included at other locations in the peritoneal dialysate generation flow path 101. A storage container can be included upstream of the sterilization module, and downstream of the water purification module. Before the fluid is utilized in the cycler stage, the fluid can be pumped through the sterilization module, eliminating issues related to storage of sterile fluid. The storage containers can be either upstream or downstream of the concentrate source 105. Concentrates can be added to fluid before storing the fluid, or after storage of the fluid but prior to sterilization in the sterilization module. The peritoneal dialysate is then pumped through valve 112 and into infusion line 118. Infusion line 118 can include a connector 114 for connection to any non-integrated cycler (not shown in FIG. 1), which can infuse the peritoneal dialysate into the patient. The direct connection to an external cycler can use any type of connector known in the art. The connectors can be single-use or reusable connectors and should provide for sterile transfer of fluids. The connectors should preferably be closed connectors, to avoid contact between the fluids and the external environment. A non-limiting example of a connector that can be used for a direct connection to an external cycler is the INTACT® connectors provided by Medinstill Development LLC, Delaware, US. Alternatively, an integrated cycler (not shown in FIG. 1) can be provided, with direct infusion of the peritoneal dialysate from the peritoneal dialysate generation flow path 101 into the peritoneal cavity of the patient.

After a dwell time in the peritoneal cavity of the patient, the peritoneal dialysate can be drained from the peritoneal cavity of the patient by the cycler and returned to the peritoneal dialysate generation flow path 101 for fluid regeneration for subsequent cycles. Connector 115 on drain line 119 can connect to a drain line of a non-integrated cycler to return the used peritoneal dialysate back to the peritoneal dialysate generation flow path 101 through valve 113. The fluid returned to the peritoneal dialysate generation flow path 101 from the peritoneal cavity of the patient can again be pumped through the peritoneal dialysate generation flow path 101 as described to regenerate the peritoneal dialysate for reuse in dialysis. A waste reservoir 122 can be fluidly connected to the peritoneal dialysate generation flow path 101 through valve 123 for removal of excess fluid drained from the peritoneal cavity of the patient.

The peritoneal dialysate regeneration system illustrated in FIG. 1 is a modular system that can be connected to any non-integrated cycler for infusion into and out of the peritoneal cavity of the patient. Alternatively, the dialysate storage container 108 can be detachable from the system, and separately connected to a non-integrated cycler. The dialysate storage container 108 can include one or more sterilized dialysate bags. The dialysate bags, once filled with peritoneal dialysate, can be stored until needed by the patient. The dialysate storage container 108 can alternatively be a reusable sterilized container or bag. The reusable container or bag can be cleaned and sterilized daily, or at set time periods. Alternatively, the dialysate storage container 108 can be any type of storage container, such as a stainless steel container. The dialysate storage container 108 can store enough peritoneal dialysate for a single infusion of peritoneal dialysate into the patient, or enough peritoneal dialysate for multiple infusions into a patient. The connectors to the dialysate storage container 108 can be any type of connector known in the art. The connectors can be single use or disposable connectors that provide transfer of sterile fluids. A non-limiting example of connectors that can be used with the described system is the Lynx®-Millipore connectors available from Merck KGaA, Darmstadt, Germany.

As described, any number of concentrate sources can be included in the peritoneal dialysate generation flow path 101. Table 3 provides exemplary non-limiting ranges of solutes that can be added from a single concentrate source 105 to the peritoneal dialysate generation flow path 101, including the starting concentration of the solutes in the concentrate source, as well as exemplary final volumes of the solutes in the dialysate and the exemplary flow rates of both the solutes and the water in the dialysate generation flow path that will achieve those concentrations. The solutes shown in Table 3 are traditional peritoneal dialysate solutes. Table 4 shows exemplary ranges of solutes that can be used as a low GDP formulation. Table 5 shows exemplary ranges of solutes that can be used with icodextrin as the osmotic agent. Icodextrin is sometimes used as an osmotic agent for a long dwell period. If dextrose or glucose is used in a long dwell period, reabsorption of the ultrafiltrate can occur, reducing the net volume of fluid removed. Icodextrin results in a long sustained ultrafiltration, and can provide improved ultrafiltration efficiency over a long dwell period. One of skill in the art will understand that the concentrations of any of the solutes shown in Tables 3-5 can be altered by altering the flow rates of the system pump 103 or concentrate pump 106. However, the ratio of the solutes included is fixed if using a single concentrate source 105. If the ratio of the solutes needs to be altered for any reason, a new concentrate solution may be needed.

TABLE 3
Exemplary solutes for addition from a single concentrate source

| | Concentration | Solution volume | Flow rate |
|---|---|---|---|
| Component | (g/l) | (ml/L) | (ml/min) |
| Glucose | 100-850 | 50-400 | 1-18 |
| Sodium Chloride | 13-108 | 50-400 | 1-18 |
| Sodium Lactate | 11-90 | 50-400 | 1-18 |
| MgCl₂•6H₂O | 0.13-1.02 | 50-400 | 1-18 |
| CaCl₂•2H₂O | 0.6-5.1 | 50-400 | 1-18 |
| Water | | 600-950 | 50-1000 |

TABLE 4
Exemplary solute ranges in a low GDP solution

| | Concentration | Solution volume | Flow rate |
|---|---|---|---|
| Component | (g/l) | (ml/L) | (ml/min) |
| Glucose | 100-900 | 50-400 | 1-18 |
| Sodium Chloride | 13-108 | 50-400 | 1-18 |
| Sodium Lactate | 11-90 | 50-400 | 1-18 |
| MgCl₂•6H₂O | 0.13-1.02 | 50-400 | 1-18 |
| CaCl₂•2H₂O | 0.6-5.1 | 50-400 | 1-18 |
| Water | | 600-950 | 50-1000 |

TABLE 5
Exemplary solute ranges in icodextrin solution

| | Concentration | Solution volume | Flow rate |
|---|---|---|---|
| Component | (g/l) | (ml/L) | (ml/min) |
| Icodextrin | 100-850 | 100-400 | 2-37 |
| Sodium Chloride | 13-108 | 100-400 | 1-18 |
| Sodium Lactate | 11-90 | 100-400 | 2-37 |
| MgCl₂•6H₂O | 0.13-1.02 | 100-400 | 2-37 |
| CaCl₂•2H₂O | 0.6-5.1 | 100-400 | 2-37 |
| Water | | 600-900 | 50-1000 |

Although using a single concentrate source in the system requires a fixed ratio of solutes in the generated peritoneal dialysate, a single concentrate source provides certain advantages. Storage requirements are decreased, as only a single concentrate solution needs to be stored for a given dialysate prescription. There is also a lower risk of patient error in adding solutes to the dialysate in the proper amounts. A single concentrate source also requires less supplies, less pumps, and less hardware. Further, because fewer containers are needed, the containers are easier to manage, clean, and disinfect. One of skill in the art will understand that a higher concentration of solutes in the concentrate source will allow minimization of the container size and maximization of the source water used in PD solution preparation, lowering costs. The limiting factor is mutual solubility of the components, which is generally limited by glucose or icodextrin solubility. The flow rate for the source water can be optimized to adjust the time required to prepare the solution. In the case of on-demand dialysate preparation, a high flow rate is desired to minimize the time needed to prepare the solution. The flow rate limit will be controlled by the metering accuracy of the concentrate pump at the rate required to match the water feed. With a single concentrate source, about 150 ml/exchange can be needed, corresponding to about 600 ml/day or 4.2 L/week. The concentrate source can be sized depending on the needs of the user, with a larger concentrate source requiring less frequent refilling.

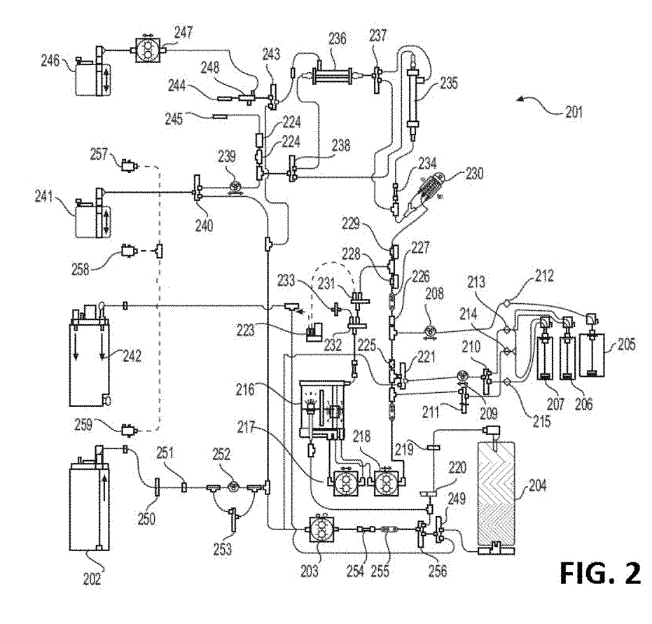

FIG. 2 illustrates a detailed flow diagram of a regenerative peritoneal dialysate system. The initial peritoneal dialysate can be generated with water from water source 202. Water pump 252 provides a driving force for moving water from water source 202 into the peritoneal dialysate generation flow path 201. Filter 250 can remove any particulate matter from the water prior to entering the peritoneal dialysate generation flow path 201. Valve 253 allows for fast priming of the sorbent cartridge 204 and the peritoneal dialysate generation flow path 201. Check valve 251 prevents back flow of fluid into the water source 202. System pump 203 provides a driving force for moving fluid through the peritoneal dialysate generation flow path 201. The water can pass through pressure sensor 254 to ensure the incoming water is within a predetermined pressure threshold. Conductivity sensor 255 measures the conductivity of the incoming water. The water passes through valves 256 and 249 and enters sorbent cartridge 204 for purification. Valve 249 can also direct fluid to waste reservoir 242 during disinfection at the beginning of each session. Filter 219 removes any particulate matter from the fluid after passing through sorbent cartridge 204. The fluid is then pumped through valve 220 and degassed in degasser 216. Degassing pump 217 provides a driving force for moving the fluid through degasser 216. Pump 218 pumps fluid back into the peritoneal dialysate generation flow path 201. Although shown as a degasser 216 with a degassing sprayer in FIG. 2, the degasser 216 can be any type of degasser known in the art. Vacuum pump 223 can create a vacuum to remove air from the top of the degassing vessel through valves 232 and 231. Vent 233 allows the air to escape.

To generate the peritoneal dialysate, solutes are added from concentrate sources 205, 206, and 207. As explained, any number of concentrate sources can be used. As illustrated in FIG. 2, the concentrate sources can include sodium chloride source 207, ion concentrate source 206, and osmotic agent source 205. Concentrate pumps 208 and 209 provide a driving force for pumping concentrates into the peritoneal dialysate generation flow path 201. Filters 212, 213, 214, and 215 can remove particulate matter from the concentrates prior to entering peritoneal dialysate generation flow path 201. Valves 210 and 211 control adding concentrates to the peritoneal dialysate generation flow path 201. Valve 221 can direct the concentrates to a location upstream of sorbent cartridge 204 during priming of the system. Conductivity sensor 225 determines the conductivity of the fluid after addition of sodium chloride and other concentrates. Mixing chamber 226 mixes the concentrates with the fluid in peritoneal dialysate generation flow path 201. Conductivity sensor 227 determines the conductivity of the generated peritoneal dialysate. Flow meters 228 and 229 determine the flow rate of the fluid after addition of the concentrates. A pH and ammonia sensor 230 determines the pH of the peritoneal dialysate, and can determine the presence of ammonia or ammonium ions that remain in the peritoneal dialysate after passing through the sorbent cartridge 204. Pressure sensor 234 measures the pressure of the fluid prior to sterilization and is used in the control circuit to control the pressure.

The generated peritoneal dialysate can be sterilized by pumping the peritoneal dialysate through a sterilization module, shown as two ultrafilters 235 and 237 in FIG. 2. The peritoneal dialysate can be pumped through a first ultrafilter 235, through three-way valve 237 and then through an optional second ultrafilter 236. Valve 238 can be used for back flushing the ultrafilters 235 and 237. The sterilized peritoneal dialysate can be pumped through valve 243 to cycler connector 244, which can connect to an infusion line of any non-integrated cycler (not shown). Alternatively, valve 248 can direct fluid into a dialysate container 246 for storage and later use. Pump 247 controls the movement of fluid into and out of dialysate container 246. As described, additional or alternative storage containers can be included upstream of the sterilization module, either before or after addition of the concentrates. Storage of the fluid prior to the sterilization module may address potential sterilization issues associated with storage of sterilized dialysate during the storage phase. Dialysate stored in dialysate container 246 can be pumped back into the peritoneal dialysate generation flow path 201 by pump 247 and into cycler connector 244 for infusion into a patient. Alternatively, dialysate container 246 can be detached from the system and separately used with any non-integrated cycler.

After the peritoneal dialysate is infused into the peritoneal cavity of a patient the peritoneal dialysate is held in the patient for a dwell period. After the dwell period, the peritoneal dialysate is drained from the patient. The fluid returned to the peritoneal dialysate generation flow path 201 from the peritoneal cavity of the patient is pumped through cycler connector 245 which can connect to a drain line of any non-integrated cycler (not shown). If a detachable dialysate container 246 is used, waste and storage reservoir 241 can be connected to collect used dialysate from the patient. After use, waste and storage reservoir 241 can be reconnected to the peritoneal dialysate generation flow path 201 and the used peritoneal dialysate pumped from the waste and storage reservoir 241 back into the peritoneal dialysate generation flow path 201. Flow meter 222 determines the amount of fluid removed from the patient, and pressure sensor 224 monitors the draw pressure when removing fluid from the patient. The used peritoneal dialysate can be removed from the patient through valve 240 by pump 239 and pumped into storage reservoir 241. The remaining used peritoneal dialysate can be pumped through the peritoneal dialysate generation flow path 201, and back through the sorbent cartridge 204 to regenerate the peritoneal dialysis. Connectors 257, 258, and 259 can be used to disinfect the peritoneal dialysate generation flow path 201.

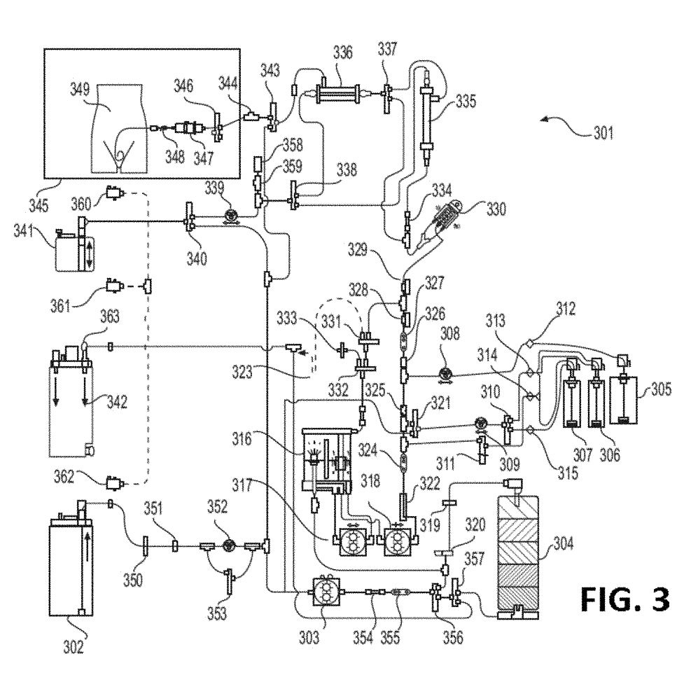

FIG. 3 illustrates a detailed flow diagram for regeneration of peritoneal dialysate with an integrated cycler 345. The initial peritoneal dialysate can be generated with water from water source 302. Water pump 352 provides a driving force for moving water from water source 302 into the peritoneal dialysate generation flow path 301. Filter 350 can remove any particulate matter from the water prior to entering the peritoneal dialysate generation flow path 301. Valve 353 allows for fast priming of the sorbent cartridge 304 and the peritoneal dialysate generation flow path 301. Check valve 351 prevents back flow of fluid into the water source 302. System pump 303 provides a driving force for moving fluid through the peritoneal dialysate generation flow path 301. The water can pass through pressure sensor 354, which monitors pressure of fluid moving into sorbent cartridge 304. If the pressure exceeds a defined pressure, the fluid flow rate can be slowed to maintain a maximum pressure. Conductivity sensor 355 measures the conductivity of the incoming fluid. The water passes through valves 356 and 357 and enters sorbent cartridge 304 for purification. Valve 356 can bypass the sorbent cartridge 304 during priming. Valve 357 can also drain fluid to waste reservoir 342 at the start of treatment. Filter 319 removes any particulate matter from the fluid after passing through sorbent cartridge 304. The fluid is then pumped through valve 320 and degassed in degasser 316. Degassing pump 317 provides a driving force for moving the fluid through degasser 316. Pump 318 can pump fluid back into the peritoneal dialysate generation flow path 301. Although shown as a degasser 316 with a degassing sprayer in FIG. 3, the degasser 316 can be any type of degasser known in the art. Vacuum pump 323 can create a vacuum to remove air from the top of the degassing vessel through valves 332 and 331. Vent 333 allows the air to escape. After exiting the degassing vessel, the fluid can be heated by inline heater 322 to a desired temperature. The heater 322 can be placed at any location in the flow path prior to delivery to the patient 349. In any embodiment, the heater 322 can be located after the exit of the sterilization module, particularly if fluid is stored prior to fluid passing through the sterilization module. Temperature sensor 324 can ensure the temperature of the fluid is within a predetermined range.

To generate the peritoneal dialysate, solutes are added from concentrate sources 305, 306, and 307. As explained, any number of concentrate sources can be used. As illustrated in FIG. 3, the concentrate sources can include sodium chloride source 307, ion concentrate source 306, and osmotic agent source 305. Concentrate pumps 308 and 309 provide a driving force for pumping concentrates into the peritoneal dialysate generation flow path 301. Filters 312, 313, 314, and 315 can remove particulate matter from the concentrates prior to entering peritoneal dialysate generation flow path 301. Valves 310 and 311 control adding concentrates to the peritoneal dialysate generation flow path 301. Valve 321 can direct the concentrates to a location upstream of sorbent cartridge 304 during priming of the system. Conductivity sensor 325 determines the conductivity of the fluid after addition of sodium chloride and other concentrates. Mixing chamber 326 mixes the concentrates with the fluid in peritoneal dialysate generation flow path 301. Conductivity sensor 327 determines the conductivity of the generated peritoneal dialysate. Flow meters 328 and 329 determine the flow rate of the fluid after addition of the concentrates. A pH and ammonia sensor 330 determines the pH of the peritoneal dialysate, and can determine the presence of ammonia or ammonium ions that remain in the peritoneal dialysate after passing through the sorbent cartridge 304. Pressure sensor 334 determines the pressure of the fluid prior to sterilization.

The generated peritoneal dialysate can be sterilized by pumping the peritoneal dialysate through a sterilization module, shown as two ultrafilters 335 and 337 in FIG. 3. The peritoneal dialysate can be pumped through a first ultrafilter 335, through three-way valve 337 and then through an optional second ultrafilter 336 and/or an optional UV light source (not shown). Valve 338 is used for back flushing the ultrafilters 335 and 337. The sterilized peritoneal dialysate can be pumped through valve 343 to the integrated cycler 345. Pressure sensor 344 ensures the peritoneal dialysate is at a safe pressure for infusion into the peritoneal cavity of the patient 349. The pump 303 can use any safe pressure for infusing fluid into the patient 349. Generally, the pump pressures are on average set at ±10.3 kPa or 77.6 mmHg. If there is no fluid flow, the maximum pressure can increase to ±15.2 kPa or 113.8 mmHg for a short period, such as less than 10 seconds. Three-way valve 346 controls the infusion and drainage of peritoneal dialysate from the patient 349. In any embodiment, an additional microbial filter (not shown) may be used to sterilize the peritoneal dialysis fluid immediately before the peritoneal dialysate enters the patient 349. Connector 347 connects to catheter 348 for infusion and drainage of the peritoneal dialysate from the patient 349. A filter (not shown) can be included between the three-way valve 346 and the catheter 348 for additional cleaning of the peritoneal dialysate. In any embodiment, the filter can be a disposable filter. Treatment, other than the first cycle of the day or night in APD or CAPD, generally begins with drainage of the peritoneal cavity of the patient 349, prior to infusing the fresh peritoneal dialysate into the patient 349. Overfill, or excessive solution in the peritoneal cavity beyond the target volume may present complications in therapy. Overfill can be caused by many factors, including failing to fully drain the peritoneal cavity prior to infusion of fresh peritoneal dialysate. In any embodiment, the integrated cycler 345 can start therapy with a drain step to ensure that no peritoneal dialysate remains in the peritoneal cavity. Monitoring both pressure and volume of peritoneal dialysate introduced to the patient 349 can avoid overfill. If the pressure rises to a certain point, the system can be programmed to end filling or send an alert to a user to complete filling of the peritoneal cavity at a desired level. The volume of peritoneal dialysate extracted from and introduced to the patient 349 can also be monitored with flow meters (not shown) to ensure proper volumes of exchanges. Draining the peritoneal cavity can be performed in a similar manner by monitoring the pressure and volume of the drained peritoneal dialysate.

After a dwell period, the peritoneal dialysate is drained from the patient 349. The fluid is returned to the peritoneal dialysate generation flow path 301 from the peritoneal cavity of the patient 349. Drain pump 339 provides a driving force for draining the peritoneal dialysate from the patient 349. There is no set rate for draining peritoneal dialysate from the peritoneal cavity of the patient 349, and any flow rate can be used with the integrated cycler 345. A slow flow is defined as a drain flow rate of less than 50 mL/min for a standard fill, and less than 15 mL/min for a low fill. No flow is defined as a drain flow rate of less than 12 mL/min for a standard fill, and less than 3 mL/min for a low fill. If the detected flow rate of the drained dialysate is below the cutoffs, the system can generate an alarm. With the online generation of fluid described, a flow rate of 300 ml/min can support an exchange time of between 10 and 15 minutes for a full cycle of draining and filling the peritoneal cavity of a patient 349. The fill/drain cycle can be performed in 10 to 15 minutes with 2 to 3 L of fluid moving in total, half of which is moved into the peritoneal cavity and half of which is moved out of the peritoneal cavity. The peritoneal cavity can be drained with a slight negative pressure of about 50 to 100 mbar created by the drain pump 339. The drain rate can be up to 300 ml/minute or greater and can vary throughout the session. For example, a drain rate can be high such as at 300 ml/min, and then slow, such as to 100 ml/min, as the cavity approaches an empty point. Similarly, a fill rate can be as high as 300 ml/min, and also vary throughout a session. In the case of power failure during treatment, the valves and pumps can be closed to prevent any dialysate flow. If power is returned quickly, the therapy can resume. With a longer power failure, an alert can be generated instructing the patient 349 to manually drain the peritoneal dialysate. In any embodiment, a battery backup can be included in the case of power failure.

The used peritoneal dialysate is pumped through three-way valve 346 and back into peritoneal dialysate generation flow path 301. Flow meter 358 determines the amount of fluid removed from the patient 349, and pressure sensor 359 monitors the draw pressure when removing fluid from the patient 349. Peritoneal dialysate can be pumped through valve 340 into storage and waste reservoir 341 for storage of the removed fluid prior to starting delivery of regenerated fluid. The storage and waste reservoir 341 can be any size, including between 3-6 L. The remaining used peritoneal dialysate can be pumped through the peritoneal dialysate generation flow path 301, and back through the sorbent cartridge 304 to regenerate the peritoneal dialysis. Connectors 360, 361, and 362 are used to disinfect the peritoneal dialysate generation flow path 301.

For automated disinfection of the system, connector 347 can be connected to connector 360 to form a flow loop. Disinfectant can be circulated through the flow loop and heated. The disinfectant can be heated to any temperature capable of disinfecting the system, including temperatures of 90° C. or greater. The disinfectant can be introduced to the flow loop and recirculated at elevated temperatures to ensure complete disinfection. The disinfectant used can be any suitable disinfectant known in the art, including peracetic acid, citric acid, or bleach. The connectors and components of the system can be gamma and autoclave compatible to resist the high temperatures used during disinfection. The system can be primed by introducing a priming fluid to the peritoneal dialysate generation flow path 301 and integrated cycler 345.

By generating and immediately using the peritoneal dialysate, the dialysate storage time can be reduced, reducing the possibility of bacterial growth. A user interface can be included on the peritoneal dialysis generation system in communication with the control system, allowing a patient 349 to direct the generation of peritoneal dialysate at a selected time as needed at a selected time. Additionally, or alternatively, the peritoneal dialysate machine can include a timer, and the timer can cause the peritoneal dialysate machine to generate peritoneal dialysate at predetermined times according to the patient's 349 peritoneal dialysis schedule. Alternatively, the peritoneal dialysate generation machine can be equipped with wireless communication, such as Wi-Fi, Bluetooth, Ethernet, or any other wireless communication system known in the art to meet patient or clinic needs. The user can direct the peritoneal dialysis machine to generate peritoneal dialysate at a specified time from any location. By using a timer, user interface, or wireless communication to control the generation of peritoneal dialysate on demand, the peritoneal dialysate storage time can be reduced, lowering the chances of generating significant amounts of degradation products or allowing bacterial growth.

The peritoneal dialysate can be generated and used in real time, with direct infusion of the peritoneal dialysate into the patient through an integrated cycler. Alternatively, the peritoneal dialysate can be generated and stored prior to use with a non-integrated cycler. For real time generation and use of the peritoneal dialysate, the flow rate of fluid through the peritoneal dialysate generation flow path can be between 50 and 300 ml/min. If a dialysate storage container is used to store generated peritoneal dialysate, the flow rate of fluid through the peritoneal dialysate generation flow path can be any rate. The integrated or non-integrated cycler and the rest of the system can communicate for the purposes of generation and use of the peritoneal dialysate by any method known in the art, including Bluetooth, Wi-Fi, Ethernet, or direct hardware connections. Additional valves and regulators (not shown in FIG. 3) can be included to aid in connection and operation of the peritoneal dialysate generation flow path and an integrated or non-integrated cycler. The integrated or non-integrated cycler and the peritoneal dialysate generation flow path can communicate directly, or can each communicate with a control system for control over the generation, regeneration, and use of the peritoneal dialysate.

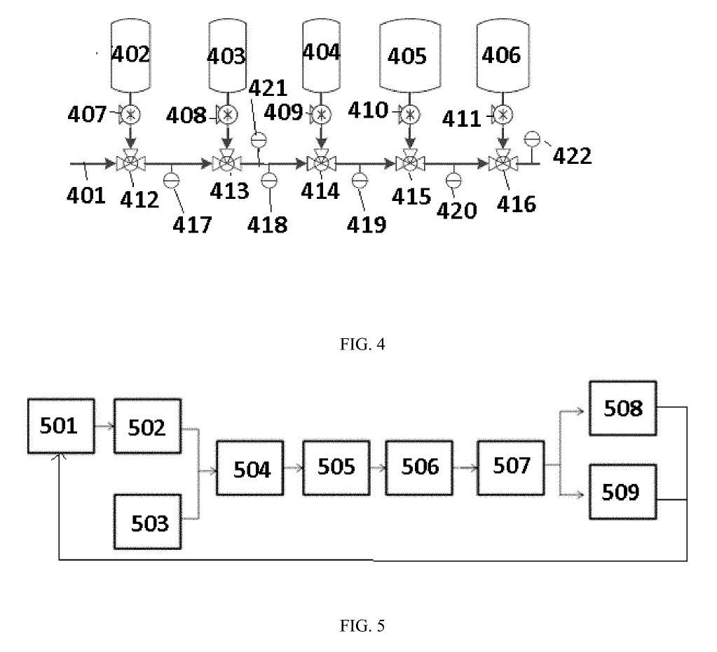

In any embodiment of the first or second aspects of the invention, solutes can be added to a peritoneal dialysate generation flow path 401 from two or more separate concentrate sources, as shown in FIG. 4. The peritoneal dialysate generation flow path 401 can be fluidly connected to a water source and a peritoneal dialysate regeneration module upstream of the concentrate sources 402-406, and fluidly connected to a sterilization module, an optional integrated cycler, and optionally a dialysate container downstream of the concentrate sources 402-406, as illustrated in FIGS. 1-3. For clarity, the components have been omitted from FIG. 4.

As illustrated in FIG. 4, the concentrate source can include one or more ion concentrate sources, such as sodium chloride source 402 containing sodium chloride to be added in a controlled addition to the peritoneal dialysate generation flow path 401 by concentrate pump 407 through valve 412, sodium lactate source 403 containing sodium lactate to be added in a controlled addition to the peritoneal dialysate generation flow path 401 by concentrate pump 408 through valve 413, magnesium chloride source 404 containing magnesium chloride to be added in a controlled addition to the peritoneal dialysate generation flow path 401 by concentrate pump 409 through valve 414, and calcium chloride source 405 containing calcium chloride to be added in a controlled addition to the peritoneal dialysate generation flow path 401 by concentrate pump 410 through valve 415. One of skill in the art will understand other ions can be used in formulation of peritoneal dialysate, and each can be contained in a separate ion concentrate source or combined into one or more combined ion concentrate sources. The concentrate source also includes one or more osmotic agent sources, such as dextrose source 406 containing dextrose to be added to the peritoneal dialysate generation flow path 401 by concentrate pump 411 through valve 416. Any of the concentrate pumps 407-411 can include flow meters to control the addition of the solutes. A glucose source and/or an icodextrin source can be used in addition to, or in place of, dextrose source 406. Multiple osmotic agents can be added to the peritoneal dialysate generation flow path 401 from one or more osmotic agent sources. One of skill in the art will understand other solutes can be used alternatively to, or in addition to, the solutes illustrated in FIG. 4. Any set of solutes used for peritoneal dialysate is within the scope of the invention. A control system in electronic communication with each of the concentrate pumps 407-411 can control the movement of fluid from the concentrate sources 402-406 to the peritoneal dialysate generation flow path 401. The amount of each of the concentrates moved into the peritoneal dialysate generation flow path 401 can be controlled to result in peritoneal dialysate having a prescribed solute concentration, as determined by a doctor or health care provider. The valves 412-416 can optionally be replaced with hose T junctions with additional components for preventing backflow into the concentrate source line if that particular line is not being used. A hose T is a fluid connector in a T-shape, with a port at each end for fluid to enter or exit the hose T. Optional sensors 417, 418, 419, and 420 ensure the solute concentration in the dialysate is at the correct level after each addition. The sensors 417-420 can be any type of sensor appropriate to confirm delivery of the concentrate, such as conductivity sensors. Optional pH sensor 421 ensures the pH is a proper level after addition of sodium lactate or other buffer. Optional refractive index meter 422 ensures the dextrose concentration in the dialysate is at the prescribed level. An additional sensor (not shown) can be included upstream of sodium chloride source 402 for sensing the conductivity of the water prior to addition of concentrates. One of skill in the art will understand that additional sensor arrangements can be used in the described system. Any number of sensors can be included to monitor the peritoneal dialysate concentration, including 1, 2, 3, 4, 5, 6, 7, or more sensors. The concentrate sources can contain the solutes in either solid, powdered, or solution form. A solid or powdered source of solutes can be dissolved by the system by drawing fluid from the peritoneal dialysate generation flow path 401 into the concentrate source to generate a solution with a known concentration, such as a saturated solution of the solutes. The resulting solution is added to the peritoneal dialysate generation flow path as explained.

Although shown as a refractive index meter 422 in FIG. 4, one of skill in the art will understand that alternative methods of measuring the osmotic agent concentration can be used. In any embodiment, enzyme-based sensors can detect the concentration of the osmotic agent in the dialysate. Enzyme based sensors use an enzyme capable of oxidizing the osmotic agent, such as glucose or dextrose. The enzyme is immobilized on an electrode and covered in a membrane through which the osmotic agent can pass. The electrode is used to electrochemically measure the change in either the oxidant, such as oxygen, or the product of glucose oxidation, such as hydrogen peroxide. Alternatively, electron transfer between the electrode and the enzyme can be detected with mediators, such as ferrocene to facilitate electron transfer. The osmotic agents can alternatively be detected through pulsed amperometric detection (PAD). PAD can detect glucose by applying a positive potential to a sample, resulting in oxidation of the glucose. The oxidation products are adsorbed onto the electrode and then desorbed by applying a more positive potential. Applying the more positive potential results in formation of an oxide layer on the electrode leading to passivation of the electrode surface. The catalytic activity of the electrode is then restored by application of a more negative potential, resulting in dissolution of the oxide layer.

Although illustrated as a single concentrate source in FIG. 1, three concentrate sources in FIGS. 2-3, and five separate concentrate sources in FIG. 4, one of skill in the art will understand that any number of concentrate sources can generate the peritoneal dialysate, including 1, 2, 3, 4, 5, 6, 7, or more concentrate sources. Any two or more of the separate concentrate sources illustrated in FIG. 4 can be combined into a single solute source, such as by combining all or some of the ion concentrate sources into a single ion concentrate source where the mixed contents do not cause precipitation of the mixed concentrates.

Although each concentrate source is illustrated in FIG. 4 with a separate concentrate pump and fluid line, one of skill in the art will understand that more than one concentrate source can use a single pump and fluid line, with valves arranged thereon for controlled addition to the peritoneal dialysate generation flow path 401.

The concentrate sources 402-406 can be single use concentrate sources or disposable concentrate sources. The disposable concentrate sources are used in a single peritoneal dialysate generation process and then disposed. Multiple use concentrate sources are used repeatedly, and refilled as necessary with the solute.

Table 6 provides exemplary, non-limiting, ranges of solutes that can be added to the peritoneal dialysate using a separate osmotic agent source, glucose in Table 6, and a separate ion concentrate source containing sodium chloride, sodium lactate, magnesium chloride, calcium chloride and sodium bicarbonate. Because the glucose is added separately from the ion concentrates, the ratio of glucose to the other solutes can be varied depending on the needs of the patient.

TABLE 6
Exemplary ranges of solutes in a two-concentrate source system

| | Concentration | Solution volume | Dialysate | |
|---|---|---|---|---|
| Component | (g/l) | (ml/L) | composition | |
| Part A | | | | |
| Glucose | 850 | 6-53 | 0.55-4.5 | g/dL |
| Part B | | | | |
| NaCl | 269 | 20 | 92 | mmol/L |
| Sodium Lactate | 84 | 20 | 15 | mmol/L |
| MgCl₂•6H₂O | 5 | 20 | 0.5 | mmol/L |
| CaCl₂•2H₂O | 18 | 20 | 2.5 | mmol/L |
| NaHCO₃ | 105 | 20 | 25 | mmol/L |

| | | |
|---|---|---|
| Water | 927-979 | 56.10 |

By using multiple concentrate sources, greater individualization and therapy customization can be achieved for each patient. With a single concentrate source, all solutes in the generated peritoneal dialysate must be present in a fixed ratio. By using more than one concentrate source, the ratio of solutes used in the peritoneal dialysate can be altered as the concentration of each of the osmotic agent and ion solutes can be individually controlled. For example, as illustrated by Table 6, with a single ion concentrate source and a single osmotic agent source, peritoneal dialysate with greater or less osmotic agent per concentration of ions can be generated, providing the ability to adjust the tonicity of the peritoneal dialysate solution independently of the electrolyte composition to meet the UF needs of any patient with a single set of solutions and allowing greater control over ultrafiltration. The ultrafiltration rate that results from using the peritoneal dialysate solutions can be altered by altering the concentration of the osmotic agent independently of the ionic solutes, or by changing the osmotic agent used. For example, typical ultrafiltration volumes using dextrose as the osmotic agent vary with the dextrose concentration of the peritoneal dialysate. With a 1.5% dextrose solution, the typical ultrafiltration volume is about 150 mL. With a 2.5% dextrose solution, the typical ultrafiltration volume is about 250 mL. With a 4.25% dextrose solution, the typical ultrafiltration volume can exceed 600 mL. For a single exchange using separate concentrate sources for the ion concentrates and the osmotic agent, about 50 mL of the ion concentrate and 150 mL of the osmotic agent may be needed, corresponding to about 200 ml/day or 1.4 L/week of the ion concentrate and 600 ml/day or 4.2 L/week of the osmotic agent.

Because the system is not limited to discrete glucose or other osmotic agent concentrations like known commercial solutions; the system can customize the peritoneal dialysate solutions to meet the ultrafiltration needs of patient as determined by a healthcare provider. As illustrated in Table 6, the glucose level in the peritoneal dialysate solution can be varied from 0.55 g/dL to 4.5 g/dL, while maintaining the electrolytes and buffer components constant, allowing the system to cover the range of glucose formulations currently offered commercially using a single Part A and Part B composition.

In any embodiment of the first or second aspects of the invention, two osmotic agent sources can be used, such as a dextrose source and an icodextrin source. With two osmotic agent sources, one could use dextrose during the daytime exchanges for CAPD and icodextrin during the night dwell to take advantage of the higher UF removal from icodextrin. Conversely, dextrose could be used during the night dwell and icodextrin for the extended daytime dwell in APD systems.

By using separate concentrate sources for each solute, complete individualization of the concentrations and ratios of solutes in the peritoneal dialysate can be achieved. Table 7 provides exemplary ranges of solutes that can be used in peritoneal dialysate as made by a system with each solute in a separate concentrate source. An advantage of using separate concentrate sources for each solute is that virtually any peritoneal dialysate solution composition can be prepared from a single set of component formulations. A system with separate concentrate sources for each solute is useful for patients whose prescriptions change periodically due to diet or other factors. Such patients would need to store multiple formulations if using only one or two concentrate sources, and the risk of errors would be increased.

TABLE 7
Exemplary dialysate composition from a multi-source system

| | | Solution | | |
|---|---|---|---|---|
| | Concentration | volume | Dialysate | |
| Component | (g/l) | (ml/L) | composition | |
| Part A: Glucose | 850 | 6-53 | 0.55-4.5 | g/dL |
| Part B: NaCl | 320 | 15-18 | 132-134 | mmol/L |
| Part C: Na Lactate | 1000 | 2-4 | 15-40 | mmol/L |
| Part D: MgCl2•6H2O | 500 | 0.2-0.4 | 0.5-1.0 | mmol/L |
| Part E: CaCl2•2H2O | 700 | 0.5-1.0 | 2.5-3.5 | mmol/L |
| Part F: NaHCO3 | 85 | 0-34 | 0-34 | mmol/L |
| Part G: Icodextrin | 1000 | 0-75 | 0-7.5 | g/dL |
| Water | | 820-971 | | |

In any embodiment, the one or more concentrate sources can be detachable from the rest of the system for sterilization. The concentrate sources can also be sterilized each time the concentrate sources are filled with new concentrate solutions. Further, the concentrate sources can be sterilized after a set number of uses, or after a set period of time. Moreover, the concentrate sources and the rest of the peritoneal dialysate generation system can be sterilized without any of the components by passing a disinfection solution, such as a citric acid, peracetic acid, or bleach solution, through all of the lines and containers of the system. Disinfectant can be circulated through the peritoneal dialysate generation system and heated. The disinfectant can be heated to any temperature capable of disinfecting the system, including temperatures of at least 90° C. or greater. The disinfectant can be introduced to the peritoneal dialysate generation system and recirculated at elevated temperatures, as needed, to ensure complete disinfection.

FIG. 5 illustrates an overview of generating peritoneal dialysate in accordance with any embodiment of the first or second aspects of the invention. Fluid from a source 501 can be purified by a peritoneal dialysate regeneration module 502, as explained. Concentrates from a single concentrate source 503, which can contain both ion concentrates and one or more osmotic agents, can be added to the purified water to generate a non-sterile peritoneal dialysate solution 504. The non-sterile peritoneal dialysate solution 504 is sterilized by a sterilization module 505, which may include an ultrafilter. As explained, the peritoneal dialysate can be further purified by additional components in the sterilization module 506, such as by ultrafiltration with a second ultrafilter, by a microbial filter, or by a UV light source, to generate a sterilized peritoneal dialysate 507. The sterilized peritoneal dialysate 507 can be stored or used by any method described herein, including by immediately infusing the peritoneal dialysate into a patient 508 with an integrated cycler, or dispensing the peritoneal dialysate into a dialysate container for later use in peritoneal dialysis 509, with either an integrated or non-integrated cycler. Regardless of the method of using the generated peritoneal dialysate, the spent peritoneal dialysate can be returned to the peritoneal dialysate generation flow path for regeneration and reuse.

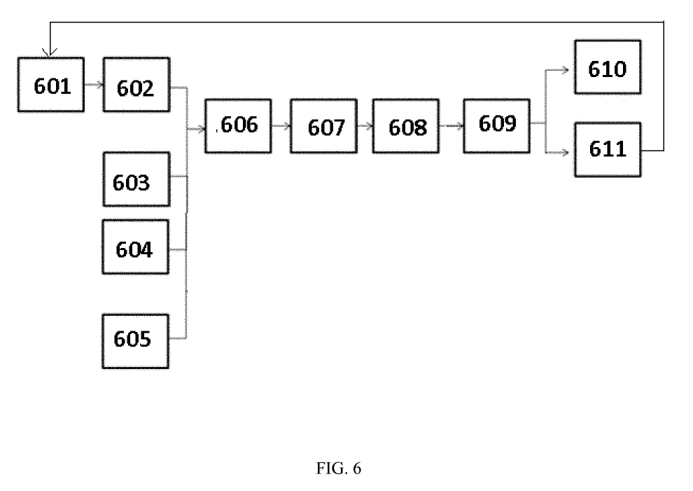

FIG. 6 illustrates an overview of generating peritoneal dialysate with multiple concentrate sources. Fluid from a source 601 can be purified by a peritoneal dialysate regeneration module 602, as explained. Concentrates from an ion concentrate source 603, which can contain sodium, magnesium, calcium, and bicarbonate, as well as any other ions to be used in peritoneal dialysis, can be added to the purified fluid. An osmotic agent, such as dextrose, can be added from a first osmotic agent concentrate source 604. A second osmotic agent, such as icodextrin, can be added from a second osmotic agent concentrate source 605. As illustrated in FIG. 4, any number of concentrate sources can be used for further individualization of the peritoneal dialysate, including separate sources for each of the ions used. After addition of the ion and osmotic agent concentrates, the fluid contains all necessary components for use in peritoneal dialysis as non-sterilized peritoneal dialysate 606. The non-sterilized peritoneal dialysate solution 606 can be sterilized by a sterilization module 607, which can include an ultrafilter or other sterilization components. The peritoneal dialysate can be further sterilized by the sterilization module 608, either by ultrafiltration with a second ultrafilter, a microbial filter, or further sterilized with a UV light source, to generate a sterilized peritoneal dialysate 609. The sterilized peritoneal dialysate 609 can be stored or used by any method described herein, including by immediately infusing the peritoneal dialysate into a patient with an integrated cycler 610, or dispensing the peritoneal dialysate into a dialysate container for later use in peritoneal dialysis with either an integrated or non-integrated cycler 611.

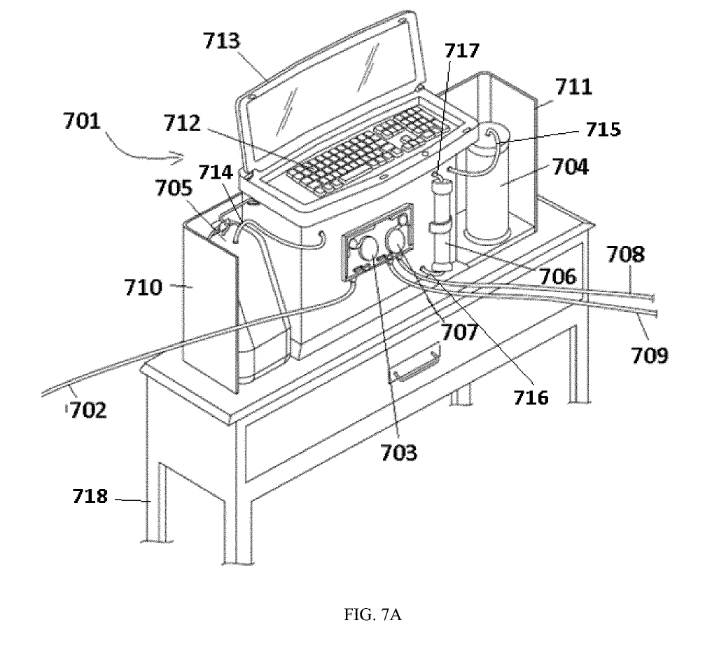

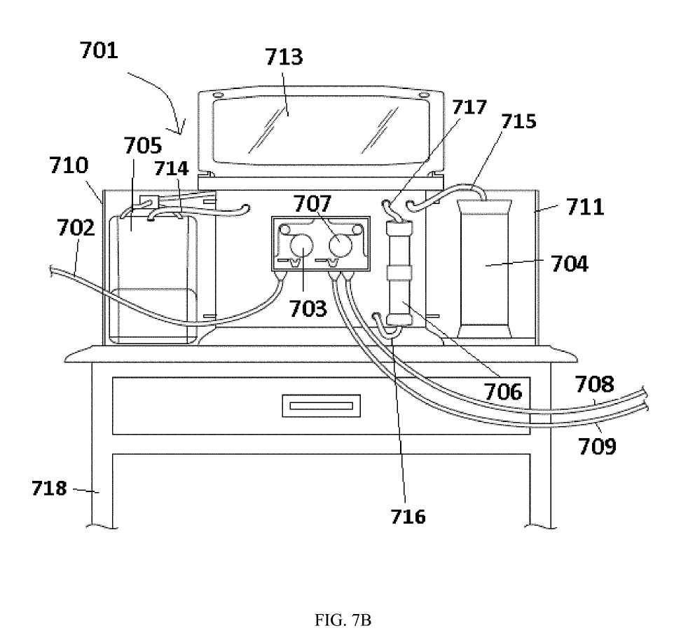

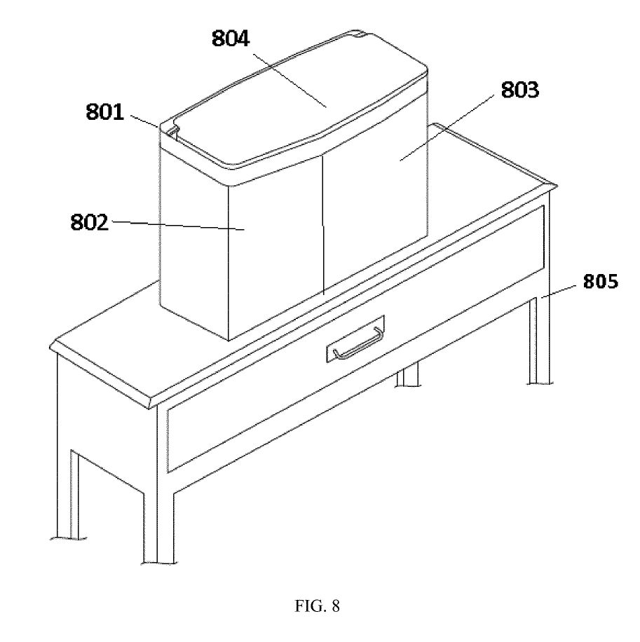

FIGS. 7A-7B illustrate a non-limiting embodiment of a peritoneal dialysate generation cabinet 701. FIG. 7A illustrates a perspective view of the peritoneal dialysate generation cabinet 701, while FIG. 7B illustrates a front view of the peritoneal dialysate generation cabinet 701. A fluid line 702 can connect a water source (not shown) to the peritoneal dialysate generation cabinet 701. System pump 707 provides a driving force for the movement of fluid throughout the peritoneal dialysate generation flow path, as described with reference to FIGS. 1-3. The water, or used peritoneal dialysate from a previous session, is pumped through the peritoneal dialysate generation cabinet 701 to a peritoneal dialysate regeneration module, shown as sorbent cartridge 704 in FIGS. 7A-B. The fluid enters the sorbent cartridge 704 through tubing (not shown) connected to the bottom of the sorbent cartridge 704 through the base of the peritoneal dialysate generation cabinet 701 and exits through tubing 715 at a top of the sorbent cartridge 704. Concentrates from concentrate source 705 are added to the fluid through tubing 714 as described to generate non-sterilized peritoneal dialysate. A concentrate pump (not shown) can provide a driving force to move fluid from the concentrate source 705 into the peritoneal dialysate generation flow path inside of the cabinet. As described, more than one concentrate source can be used. The generated peritoneal dialysate is then pumped through a sterilization module, shown as ultrafilter 706, for sterilization. The peritoneal dialysate enters the ultrafilter 706 through tubing 716 in a base of the ultrafilter 706 and exits through tubing 717 at a top of the ultrafilter 706. A second ultrafilter, microbial filter, and/or UV light source (not shown in FIG. 7) can also be included. The peritoneal dialysate is then heated by a heater (not shown in FIG. 7) and pumped into the peritoneal cavity of a patient through infusion line 708 by metering pump 703. Alternatively, infusion line 708 can be connected to any non-integrated cycler. After a dwell period, the peritoneal dialysate is drained from the patient through drain line 709 and back into the peritoneal dialysate generation flow path for regeneration. The patient tubing connected to infusion line 708 and drain line 709 can be a consumable or disposable patient tubing set, which can be replaced after each use. As described, the peritoneal dialysate generation flow path can include various sensors (not shown) for detection of conductivity, pH, refractive index, temperature, or other dialysate parameters. The sensors can be included either inside or outside of the body of the peritoneal dialysate generation cabinet 701. The fluid lines and valves connecting the components of the peritoneal dialysate generation flow path can likewise be positioned inside of the cabinet body. As described, peritoneal dialysate generation cabinet 701 can have a graphical user interface including screen 713 and keyboard 712. Messages from the control system to the user, or from the user to the control system, can be generated and read through the graphical user interface. The user can direct the generation of peritoneal dialysate through keyboard 712, and can receive messages from the system through screen 713. The system can generate alerts to the user, including any problems detected by any of the sensors, as well as the progress of peritoneal dialysate generation. Any type of user interface can be used in place of the keyboard 712 and screen 713 in FIGS. 7A-B. Alternatively, other interfaces can be included, such as lights, dials, buttons, switches or the like. In any embodiment, a single button can be used for directing the generation of peritoneal dialysate in place of the keyboard 712. In any embodiment, either keyboard 712 or screen 713 can be used alone, as with a single touch screen for both data entry and display to enable simple operation. Although shown on table 718, the peritoneal dialysate generation cabinet 701 can be used on any stable flat surface.

FIG. 8 illustrates the peritoneal dialysate generation cabinet 801 after being closed. If not in use, the concentrate source, the sorbent cartridge, and the tubing to and from the patient or non-integrated cycler (not shown in FIG. 8) can be removed, and the doors 802 and 803 of the peritoneal dialysate generation cabinet 801 can be closed to minimize storage space. Additionally, the screen 804 of the graphical user interface, as illustrated in FIGS. 7A and 7B, can be folded down into the top of the peritoneal dialysate generation cabinet 801, further minimizing storage space. The doors 802 and 803 can be open and closed by any method known in the art, including magnets, handles, indentations, hooks, or any other method of opening and closing the doors 802 and 803. The peritoneal dialysate generation cabinet 801 can have a small size and portability optimized for in-home or beside use. Although shown on table 805, the peritoneal dialysate generation cabinet 801 can be used on any stable flat surface.

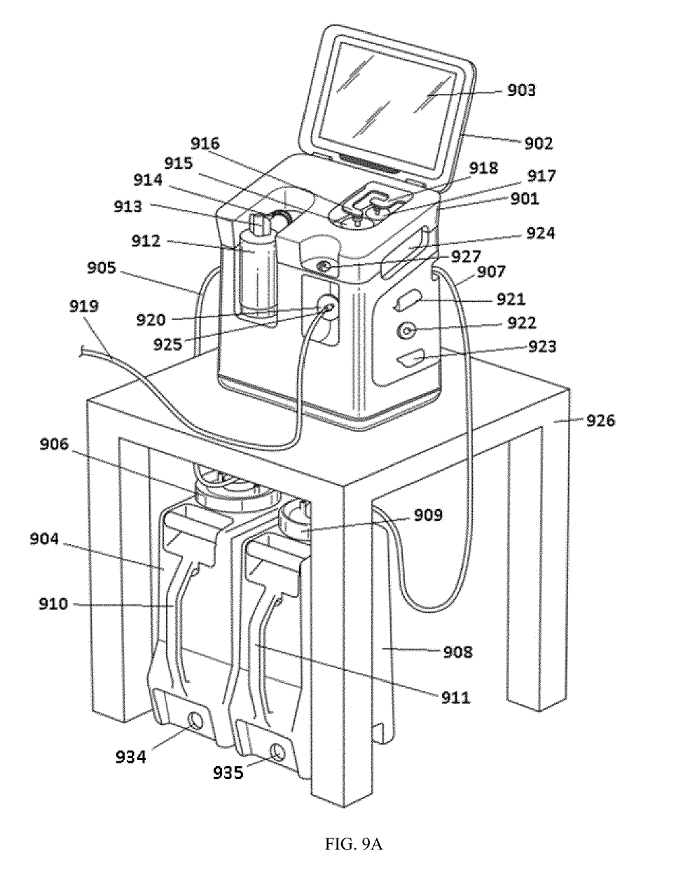

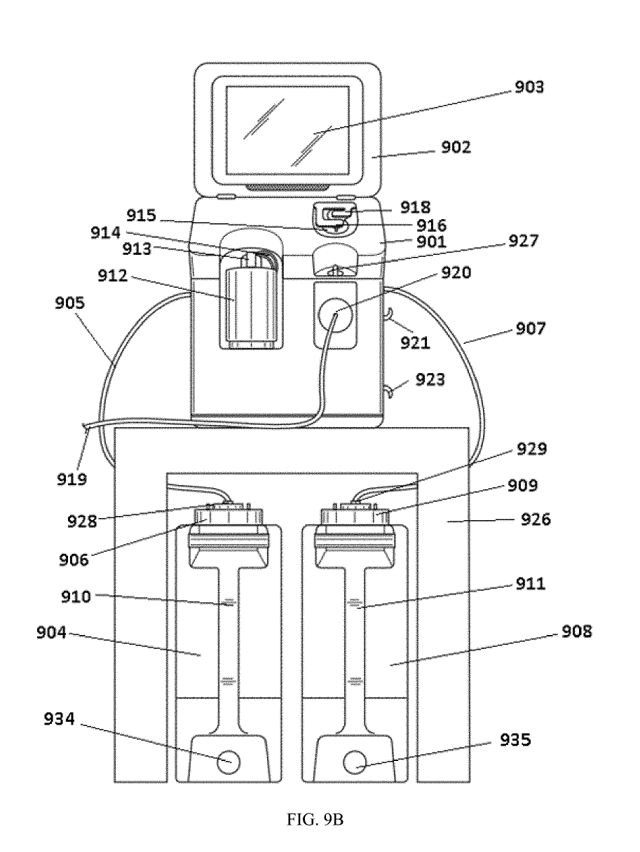

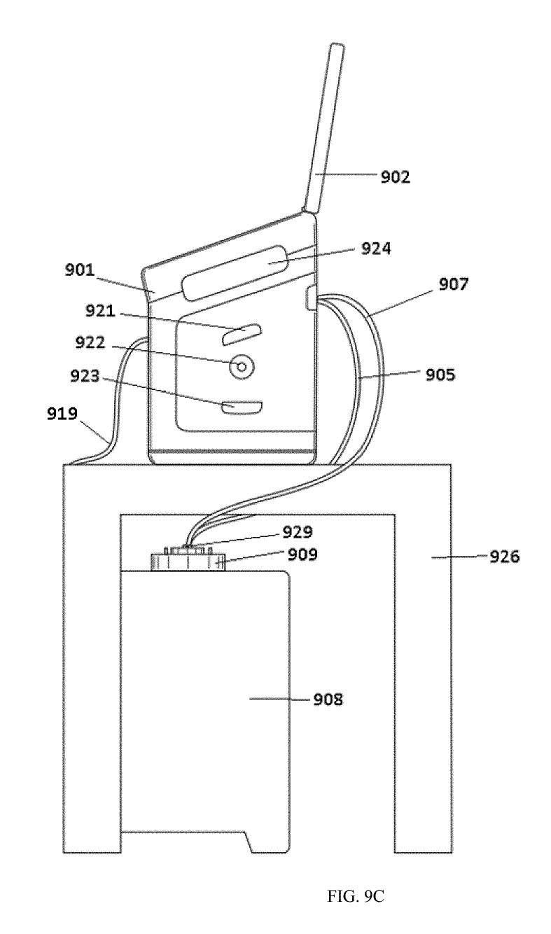

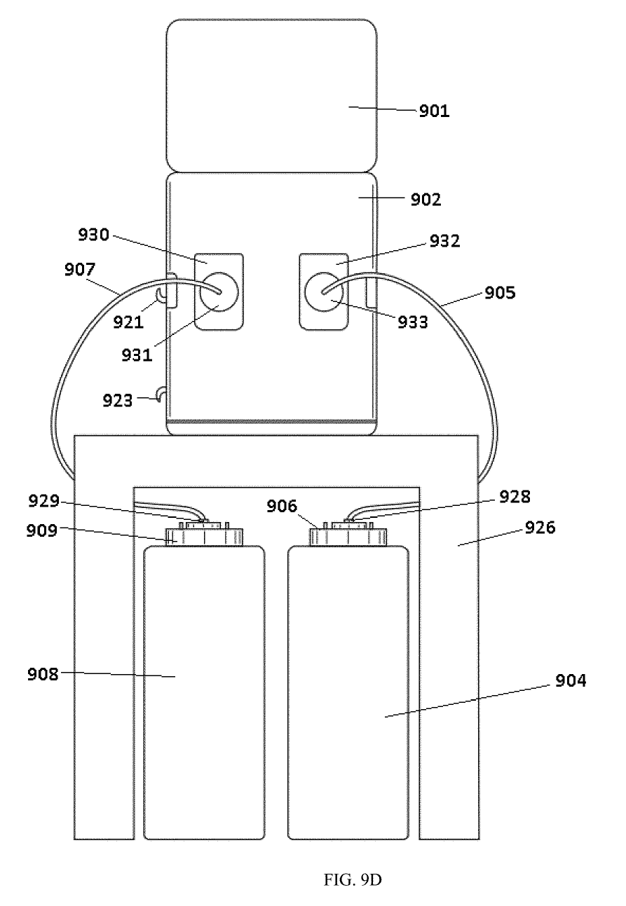

FIGS. 9A-D illustrate a non-limiting embodiment of the peritoneal dialysate generation system arranged as a peritoneal dialysate generation cabinet 901. FIG. 9A illustrates a perspective view of the peritoneal dialysate generation cabinet 901, FIG. 9B illustrates a front view of the peritoneal dialysate generation cabinet 901, FIG. 9C illustrates a side view of the peritoneal dialysate generation cabinet 901, and FIG. 9D illustrates a back view of the peritoneal dialysate generation cabinet 901.

A fluid line 905 can connect a water source 904 to the peritoneal dialysate generation cabinet 901. The fluid line 905 can enter through a connector 928 in a top 906 of the water source 904. The fluid line 905 connects to the peritoneal dialysate generation flow path as described with reference to FIGS. 1 and 5-6 through a back of the peritoneal dialysate generation cabinet 901 through connector 932 having a fitting 933 for holding the fluid line 905, as illustrated in FIG. 9D. Any of the fluid lines illustrated can be disconnected and removed from the system for cleaning and replacement. A pump (not shown) can provide a driving force for the movement of fluid throughout the peritoneal dialysate generation flow path if required. Water is pumped through the peritoneal dialysate generation cabinet 901 to a water purification module, shown as sorbent cartridge 912 in FIGS. 9A-B. The water can enter the sorbent cartridge 912 through tubing (not shown) connected to the bottom of the sorbent cartridge 912 within the peritoneal dialysate generation cabinet 901. The water exits the sorbent cartridge 912 through connector 913 and tubing 914. An osmotic agent from osmotic agent source 915 and an ion concentrate from an ion concentrate source 917 are added to the fluid as described to generate non-sterilized peritoneal dialysate. The osmotic agent concentrate is added to the fluid through paddle connector 916. The ion concentrate is added to the fluid through paddle connector 918. A concentrate pump (not shown) can provide a driving force to move fluid from the concentrate sources 915 and 917 into the peritoneal dialysate generation flow path inside of the peritoneal dialysate generation cabinet 901. As described, the system can use a single ion concentrate source in place of the two sources shown in FIGS. 9A-B, or more than two concentrate sources. The generated peritoneal dialysate can then be pumped through a sterilization module (not shown), such as an ultrafilter. A second ultrafilter and/or a UV light source can also be included. An integrated cycler (not shown in FIGS. 9A-D) can then pump the dialysate into infusion line 919 through connector 920 and into the patient. Fitting 925 allows the infusion line 919 to be removed from the system for cleaning or replacement. Waste fluids can be pumped out of the system through waste line 907, which connects to the peritoneal dialysate generation cabinet 901 through connector 930 having fitting 931. A separate drain line for returning used dialysate from the patient (not shown in FIGS. 9A-D) can also connect to the peritoneal dialysate generation cabinet 901 for regeneration of the used dialysate. A waste line 907 disposing of waste fluids enters waste container 908 through a connector 929 in the top 909 of the waste container 908. Handles 910 and 911 can be included on water source 904 and waste container 908 for easy movement and storage. Although the peritoneal dialysate generation cabinet 901 is illustrated on top of table 926 in FIGS. 9A-D, the peritoneal dialysate generation cabinet 901 can be used on any stable flat surface.

As described, the peritoneal dialysate generation flow path can include various sensors for detection of conductivity, pH, refractive index, or other dialysate parameters. The sensors can be included either inside or outside of the body of the peritoneal dialysate generation cabinet 901. The fluid lines and valves connecting the components of the peritoneal dialysate generation flow path can likewise be positioned inside of the cabinet body. As described, a top of the peritoneal dialysate generation cabinet 901 can have a graphical user interface 902 including screen 903. Messages from the control system to the user, or from the user to the control system, can be generated and read through the graphical user interface 902. The user can direct the generation of peritoneal dialysate through the graphical user interface 902, and can receive messages from the system through screen 903. The system can generate alerts to the user, including any problems detected by any of the sensors, as well as the progress of peritoneal dialysate generation. A handle 924 can be included for opening the peritoneal dialysate generation cabinet 901 to allow access to components on the inside of the cabinet. Handles 921 and 923 can be included to hold the fluid lines and power cord when not in use.

Disinfection connector 922 illustrated in FIGS. 9A and 9C can be included for disinfection of the waste line 907. During disinfection, the waste line 907 can be disconnected from waste container 908 and connected to disinfection connector 922. Disinfectant solution from a disinfectant source (not shown in FIGS. 9A-D) can then be circulated through the waste line 907 to disinfect the waste line 907. Disinfection connector 927 can be included for disinfection of fluid line 905. Fluid line 905 can be connected to disinfection connector 922 and disinfection solution can be circulated through the fluid line 905. Drain 934 on water source 904 and drain 935 on waste container 908, allow the water source 904 and waste container 908 to be drained without inverting the containers.

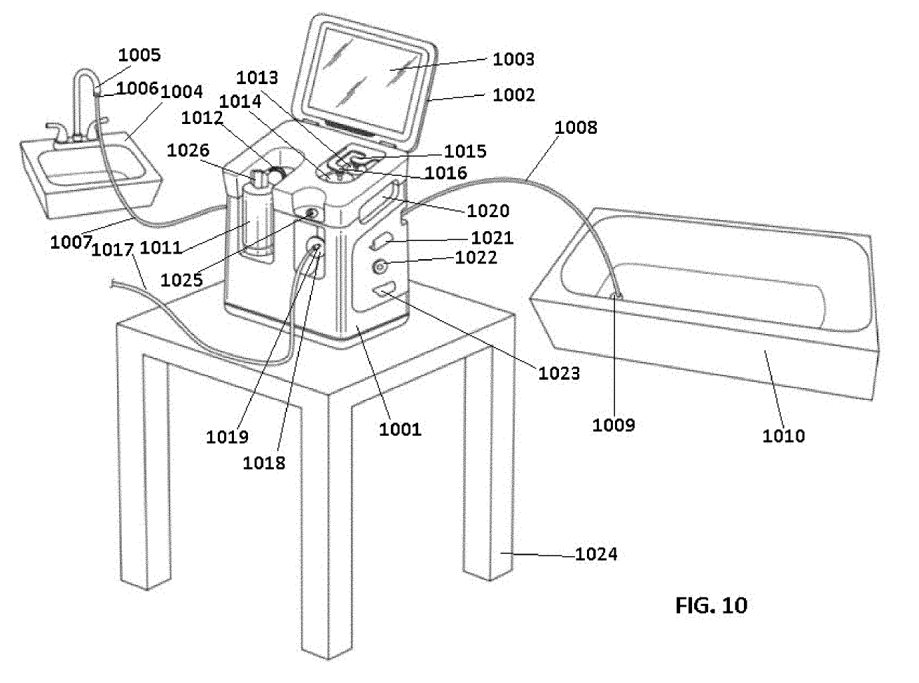

FIG. 10 illustrates a peritoneal dialysate generation cabinet 1001 using a non-purified water source, faucet 1005 in sink 1004. Although illustrated as faucet 1005 and sink 1004, one of ordinary skill in the art will understand that any water source can be used. The ability to use municipal or other non-purified sources of water allow the peritoneal dialysate generation system to work at a patient's home without the need to store large amounts of purified water or dialysate. Fitting 1006 connects the water line 1007 to the faucet 1005 or other water source, allowing the water line 1007 to be connected or disconnected as necessary. A pump (not shown) can provide a driving force for the movement of fluid throughout the peritoneal dialysate generation flow path as described with respect to FIGS. 1 and 5-6. The water is pumped through the peritoneal dialysate generation cabinet 1001 to a water purification module, shown as sorbent cartridge 1011 in FIG. 10. The water enters the sorbent cartridge 1011 through tubing (not shown) connected to the bottom of the sorbent cartridge 1011 within the peritoneal dialysate generation cabinet 1001. The water exits the sorbent cartridge 1011 through connector 1026 and tubing 1012. An osmotic agent from osmotic agent source 1013 and an ion concentrate from an ion concentrate source 1014 are added to the fluid as described to generate non-sterilized peritoneal dialysate. The osmotic agent concentrate is added to the fluid through paddle connector 1016. The ion concentrate is added to the fluid through paddle connector 1015. A concentrate pump (not shown) can provide a driving force to move fluid from the concentrate sources 1013 and 1014 into the peritoneal dialysate generation flow path inside of the peritoneal dialysate generation cabinet 1001. As described, the system can use a single ion concentrate source in place of the two sources shown in FIG. 10, or more than two concentrate sources. The generated peritoneal dialysate can then be pumped through a sterilization module (not shown), such as an ultrafilter. A second ultrafilter and/or a UV light source can also be included. An integrated cycler (not shown in FIG. 10) can then pump the dialysate into infusion line 1017 through connector 1018 and into the patient. Fitting 1019 allows the infusion line 1017 to be removed from the system for cleaning or replacement. Waste fluids can be pumped out of the system through waste line 1008, which can connect to a drain 1009 shown in bathtub 1010. A separate drain line (not shown) from the patient can be included to return used dialysate to the peritoneal dialysate generation cabinet 1001 for regeneration. Waste fluids can be disposed of in drain 1009. Although shown as a bathtub drain 1009 in FIG. 10, the waste fluids can be conveyed to any type of drain, or alternatively to a waste container as illustrated in FIGS. 9A-D. Although the peritoneal dialysate generation cabinet 1001 is illustrated on top of table 1024 in FIG. 10, the peritoneal dialysate generation cabinet 1001 can be used on any stable flat surface. In certain embodiments, the peritoneal dialysate generation cabinet 1001 and the patient can be in the same room as the water source and drain 1009. Alternatively, the patient and/or peritoneal dialysate generation cabinet 1001 can be in a separate room, with tubing long enough to reach patient. For longer distances, the tubing should be strong enough to withstand the pressures necessary in pumping fluid over longer distances.

As described, a top of the peritoneal dialysate generation cabinet 1001 can have a graphical user interface 1002 including screen 1003. Messages from the control system to the user, or from the user to the control system, can be generated and read through the graphical user interface 1002. The user can direct the generation of peritoneal dialysate through the graphical user interface 1002, and can receive messages from the system through screen 1003. The system can generate alerts to the user, including any problems detected by any of the sensors, as well as the progress of peritoneal dialysate generation. A handle 1020 can be included for opening the peritoneal dialysate generation cabinet 1001 to allow access to components on the inside of the cabinet. Handles 1021 and 1023 can be included to hold the fluid lines and power cord when not in use.

Disinfection connector 1022 can be included for disinfection of the waste line 1008. During disinfection, the waste line 1008 can be disconnected from the drain 1009 and connected to disinfection connector 1022. Disinfectant solution from a disinfectant source (not shown in FIG. 10) can then be circulated through the waste line 1008 to disinfect the waste line 1008. Disinfection connector 1025 can be included for disinfection of water line 1007. The water line 1007 can be disconnected from faucet 1005 and connected to disinfection connector 1025. Disinfectant solution can be circulate through the water line 1007 for disinfection.

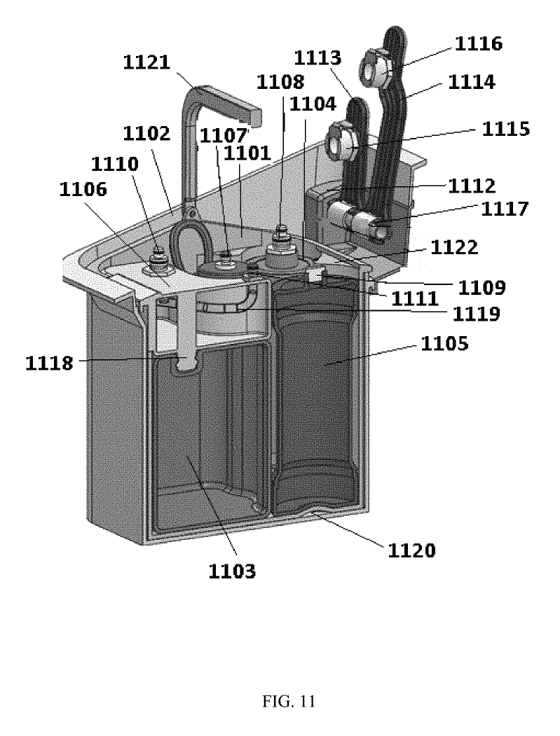

In any embodiment of the first or second aspects of the invention, the solute sources included in the dialysate generation module can be provided in a dialysis caddy. A dialysis caddy is a container adapted to contain one or more other containers, each having one or more solute sources. One non-limiting example of a dialysis caddy is shown in FIG. 11. The dialysis caddy 1101 can contain some or all of the solute sources necessary for peritoneal dialysis. In any embodiment of the first or second aspects of the invention, the dialysis caddy 1101 can contain an ion concentrate source 1103, osmotic agent source 1104, and sodium chloride source 1105. As explained, the ion concentrate source 1103 can contain any one or more of ion concentrates, such as magnesium chloride, calcium chloride or potassium chloride, or any other solutes used in peritoneal dialysis. Osmotic agent source 1104 can contain one or more osmotic agents, such as glucose, dextrose, or icodextrin. One of skill in the art will understand that any of the solutes can be contained in separate sources, and that the dialysis caddy 1101 can be adapted for any number of concentrate sources. In use, the dialysis caddy 1101 can be placed in a receiving slot of a dialysis system 1102. As shown in FIG. 11, the dialysis caddy 1101 can be configured so each of the ion concentrate source 1103, osmotic agent source 1104, and sodium chloride source 1105 are aligned with connectors for connection to the peritoneal dialysate generation flow path, such as the connectors on paddle assemblies 1113 and 1114. In any embodiment of the first or second aspect of the invention, the dialysis caddy 1101 can also contain a disinfectant source 1106, which may contain a disinfectant, such as citric acid. To disinfect the system, the dialysis caddy 1101 can be turned so container connectors 1110 and 1111 on the disinfectant source 1106 can connect to the connectors on paddle assemblies 1113 and 1114.

If the dialysis caddy 1101 is configured to generate peritoneal dialysate, container connector 1107 on ion concentrate source 1103 and container connector 1108 on osmotic agent source 1104 can connect to caddy connectors 1115 on paddle assembly 1113 and caddy connector 1116 on paddle assembly 1114. Container connector 1109 on sodium chloride source 1105 can also connect to a caddy connector (not shown in FIG. 11). The paddles can form a part of paddle assembly 1112. To connect the sources to the paddles, the paddles can be rotated downward on hinge 1117 and the caddy connectors 1115 and 1116 can connect to ion concentrate source 1103 and osmotic agent source 1104 respectively. In any embodiment of the first or second aspects of the invention, as shown in FIG. 11, the dialysis caddy 1101 and the sources within the caddy have one or more fitting feature to ensure the sources are connected to the correct paddle. The fitting features can also have the additional benefit of ensuring a tight fit within the dialysis caddy 1101, and resist inadvertent movement. The one or more fitting features can ensure each source occupies a unique position within the dialysis caddy 1101. Moreover, in any embodiment, the interior of the dialysis caddy 1101 can itself be a shaped fitting feature so each source can only be placed within a specific position or receiving compartment within the dialysis caddy 1101. In any embodiment of the first or second aspects of the invention, fitting features can be included on any connection surface of the caddy, where any source contacts the interior of the caddy. The shape of a caddy surface can include fitting feature protrusion 1120, which is a protrusion on the base of the dialysis caddy 1101. The base of sodium chloride source 1105 can be designed with a corresponding complementary indentation, such as a similarly sized recess, while the other sources lack a complimentary indentation. Sodium chloride source 1105 will be the only source that can properly fit into the position in the caddy above the fitting feature of protrusion 1120. Similarly, fitting feature protrusion 1122 is a protrusion in the side of the dialysis caddy 1101 interior. The protrusion 1122 separates the sidewall of the dialysis caddy 1101 interior into two sections. Osmotic agent source 1104 can be the only source with the proper size, shape, or geometry to fit within one of the sections on the sidewall, whereas sodium chloride source 1105 can be the only source with the proper size, shape, or geometry to fit within the other section. Each concentrate source can be positioned in one particular location within the dialysis caddy 1101. In any embodiment, the concentrate sources themselves can have fitting features to ensure the proper arrangement of the concentrate sources within the dialysis caddy 1101. In FIG. 11 disinfectant (e.g. citric acid) source 1106 includes flange 1118. Ion concentrate source 1103 has a corresponding slot. The disinfectant (e.g. citric acid) source 1106 can only be placed within the dialysis caddy 1101 at the precise position above ion concentrate source 1103. By sizing and shaping the interior of the cavity and the concentrate sources, the concentrate sources can only be placed within the dialysis caddy 1101 in a single arrangement. If the dialysis caddy 1101 is attached to the rest of the dialysis system 1102, the concentrate sources and connectors line up with the proper paddles for connection to the dialysis system, ensuring the proper solutes from the concentrate sources enter the dialysate flow path at the correct locations. The alignment also ensures the proper pumps and valves are controlling the correct solute additions. In any embodiment of the first or second aspects of the invention, handle 1121 can be included for easy of carrying and removal of the dialysis caddy 1101 from the dialysis system 1102. During use, fluid lines, such as line 1119 in disinfectant (e.g. citric acid) source 1106, can move fluids from the concentrate sources into the paddles.

Alternatively, any method of loading the peritoneal dialysate concentrates can be included in the described systems. For example, the peritoneal dialysate concentrates can be added using a disposable cassette. The disposable cassette can be multi-use or single-use with disposal of the cassette after therapy.

The connectors can include connectors for connection to reservoirs, containers, or a tap or faucet. The connectors can be any type of connector that can form a seal with a container, tap, or faucet that serve as the fluid sources in the system. The connectors can be screw-type connectors that screw onto the containers, faucet or tap, snap-type connectors that snap onto the containers, faucet, or tap, or any other type of connector known in the art. O-rings or other sealing members can be included in the connectors to form a water-tight seal with the containers, faucet, or tap.

For connection to a tap or faucet, the connectors should be able to form a seal with standard at-home faucets. The connectors can include an adjustable bore, wherein the size of the opening of the connector for connection to the tap or faucet can be increased or decreased to adjust to different size faucets. Nuts, screws, or other tightenable components can be included on the sides of the connectors allowing a user to tighten the connector around the faucet or tap regardless of the circumference of the faucet or tap. An o-ring or other sealing member can be placed on the faucet or tap to increase the effectiveness of the seal formed with the connectors.

Alternatively, a fitting can be screwed onto, or otherwise affixed to the faucet with a male end of the fitting extending outwardly from the faucet. The male end of the fitting can be inserted into the water line, and secured with an adjustable bolt, wire, or other tightening mechanism to ensure a proper seal.

For connection to a drain as illustrated in FIG. 9, the tubing for carrying waste fluids can simply be placed into the drain, bathtub, or other receptacle containing a drain for disposal. Alternatively, the tubing can include a connector for forming a sealable connection to a drain, ensuring that all waste fluids are directed into the drain.

One skilled in the art will understand that various combinations and/or modifications and variations can be made in the described systems and methods depending upon the specific needs for operation. Moreover, features illustrated or described as being part of an aspect of the invention may be used in the aspect of the invention, either alone or in combination, or follow a preferred arrangement of one or more of the described elements.

1. A system, comprising:
a water source;
a peritoneal dialysate generation flow path; wherein the peritoneal dialysate generation flow path is fluidly connectable to the water source;
one or more peritoneal dialysate regeneration modules fluidly connectable to the peritoneal dialysate generation flow path;
a concentrate source fluidly connectable to the peritoneal dialysate generation flow path; the concentrate source containing one or more solutes; and
a sterilization module fluidly connectable to the peritoneal dialysate generation flow path.

2. The system of claim 1, wherein the peritoneal dialysate generation flow path comprises connectors for connection to a cycler.

3. The system of claim 1, further comprising an integrated cycler; the integrated cycler comprising a pump, an infusion line, and a drain line; wherein the infusion line is fluidly connected to the peritoneal dialysate generation flow path downstream of the sterilization module; and wherein the drain line is fluidly connected to the peritoneal dialysate generation flow path upstream of the peritoneal dialysate regeneration module.

4. The system of claim 1, further comprising one or more dialysate containers fluidly connectable to the peritoneal dialysate generation flow path downstream of the sterilization module.

5. The system of claim 1, wherein the concentrate source comprises one or more of an osmotic agent source and an ion concentrate source.

6. (canceled)

7. (canceled)

8. The system of claim 5, wherein the osmotic agent sources contain osmotic agents selected from the group consisting of dextrose, icodextrin, amino acids, and glucose; and wherein the ion concentrate source comprises one or more from the group consisting of sodium chloride, sodium lactate, magnesium chloride, calcium chloride, potassium chloride, and sodium bicarbonate.

9. (canceled)

10. (canceled)

11. (canceled)

12. The system of claim 1, further comprising a control system for controlling one or more pumps and valves to control movement of fluid through the system; wherein the control system either (a) comprises a timer, wherein the timer causes the control system to generate peritoneal dialysate at a predetermined time; and/or (b) comprises a user interface, wherein the user interface causes the control system to generate peritoneal dialysate at a selected time.

13. (canceled)

14. (canceled)

15. The system of claim 1, wherein the sterilization module comprises one or more from the group consisting of one or more ultrafilters, a UV light source, a heater, a flash pasteurization module, a microbial filter; and combinations thereof.

16. The system claim 15, wherein the sterilization module comprises either or both of: a UV light source positioned downstream of an ultrafilter; and/or at least two ultrafilters.

17. The system of claim 1, wherein the peritoneal dialysate regeneration module comprises one or more selected from the group consisting of a sorbent cartridge, activated carbon, a reverse osmosis module, a carbon filter, an ion exchange resin, and a nanofilter.

18. (canceled)

19. (canceled)

20. (canceled)

21. (canceled)

22. A method, comprising the steps of:
pumping fluid through a peritoneal dialysate generation flow path comprising a peritoneal dialysate regeneration module;
adding one or more concentrate solutions to the fluid in the peritoneal dialysate generation flow path; and
pumping the fluid through a sterilization module.

23. The method of claim 22, wherein the fluid is fluid returned to the peritoneal dialysate generation flow path from a peritoneal cavity of a patient.

24. The method of claim 22, further comprising the steps of:
heating the fluid;
pumping the fluid into a peritoneal cavity of a patient with an integrated cycler; and
pumping the fluid from the peritoneal cavity of the patient into the peritoneal dialysate generation flow path.

25. The method of claim 22, further comprising the step of pumping the fluid into one or more dialysate containers and pumping the fluid from the one or more dialysate containers into a peritoneal cavity of a patient.

26. The method of claim 22, wherein the step of adding one or more concentrate solutions to the fluid comprises adding at least an osmotic agent and an ion concentrate to the fluid; wherein the osmotic agent and ion concentrate are added to the fluid from either a single concentrate source or from separate concentrate sources.

27. (canceled)

28. (canceled)

29. The method of claim 26, wherein the osmotic agent is one or more selected from the group consisting of glucose, dextrin, and icodextrin; and wherein the ion concentrate is added from an ion concentrate source and comprises one or more from the group consisting of sodium chloride, sodium lactate, magnesium chloride, calcium chloride, potassium chloride, and sodium bicarbonate.

30. The method of claim 26, wherein the osmotic agent comprises multiple osmotic agents; and wherein either the multiple osmotic agents are added from a single osmotic agent source; or each of the multiple osmotic agents are added from separate osmotic agent sources.

31. (canceled)

32. (canceled)

33. (canceled)

34. The method of claim 26, wherein each of the ion concentrates are added to the fluid from a single ion concentrate source; or wherein the ion concentrate source comprises multiple ion concentrate sources; and wherein each of the multiple ion concentrate sources comprise different solutes.

35. (canceled)

36. (canceled)

37. (canceled)

38. The method of claim 22, wherein the peritoneal dialysate regeneration module comprises one or more selected from the group consisting of a sorbent cartridge, activated carbon, a reverse osmosis module, a carbon filter and a nanofilter.

39. The method of claim 22, wherein the sterilization module comprises one or more from the group consisting of one or more ultrafilters, a UV light source, a microbial filter, and combinations thereof.

40. (canceled)
